(19) United States
(12) Patent Application Publication
(10) Pub. No.: US 2005/0043341 A1
(43) Pub. Date: Feb. 24, 2005

(54) 3-'Hydroxy-(-4-trifluoromethylphenyl)-methyl-7-spirocyclobutyl-5,6,7 8-tetrahydroquinolin-5-ol derivatives and the use of the same as cholesterol ester transfer protein (cetp) inhibitors

(76) Inventors: Heike Gielen, Leverkusen (DE); Siegfried Goldmann, Wuppertal (DE); Jorg Keldenich, Wuppertal (DE); Holger Paulsen, Wuppertal (DE); Carsten Schmeck, Wuppertal (DE); Stephan Siegel, Wuppertal (DE); Hilmar Bischoff, Wuppertal (DE); Martin Raabe, Wuppertal (DE); Delf Schmidt, Wuppertal (DE); Christiane Faeste, Oslo (NO)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/491,465

(22) PCT Filed: Sep. 18, 2002
(86) PCT No.: PCT/EP02/10444

(30) Foreign Application Priority Data
Oct. 1, 2001 (DE) 101484364

(51) Int. Cl. A61K 31/4747

(52) U.S. Cl. 514/278; 546/18

(57) ABSTRACT

The application relates to substituted tetrahydroquinoline derivatives, processes for their preparation and their use in medicaments.

The present invention relates to substituted tetrahydroquinolines, processes for their preparation and their use in medicaments.

Tetrahydroquinolines having pharmacological activity are disclosed in EP-A-818 448, WO 99/15504 and WO 99/1421. Substituted tetrahydronaphthalenes having pharmacological activity are disclosed in WO 99/14174.

The present invention relates to new tetrahydroquinolines of the general formula (I) 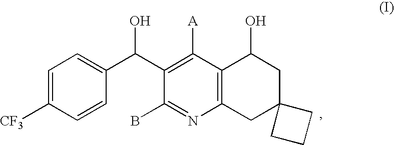
in which
A represents a radical 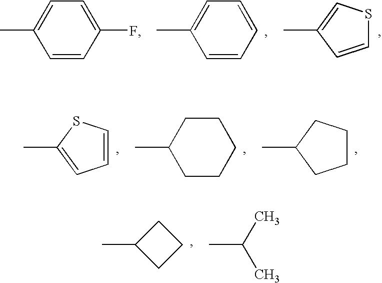
   —(CH₂)₂CH₃ and
B represents a radical

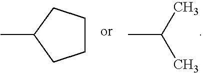

Preferred compounds of the formula (I) are those in which A represents para-fluorophenyl.

Preferred compounds of the formula (I) are likewise those in which B represents isopropyl.

The tetrahydro-quinolines according to the invention can also be present in the form of their salts. In general, salts with organic or inorganic bases or acids may be mentioned here.

In the context of the present invention, physiologically acceptable salts are preferred. Physiologically acceptable salts of the compounds according to the invention can be salts of the substances according to the invention with mineral acids, carboxylic acids or sulphonic acids. Particularly preferred salts are, for example, those with hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, propionic acid, lactic acid, tartaric acid, citric acid, fumaric acid, maleic acid or benzoic acid.

Physiologically acceptable salts can likewise be metal or ammonium salts of the compounds according to the invention which have a free carboxyl group. Those particularly preferred are, for example, sodium, potassium, magnesium or calcium salts, and also ammonium salts which are derived from ammonia, or organic amines, such as, for example, ethylamine, di- or triethylamine, di- or triethanolamine, dicyclohexylamine, dimethylaminoethanol, arginine, lysine, ethylenediamine or 2-phenylethylamine.

The compounds according to the invention can exist in stereoisomeric forms which either behave as image and mirror image (enantiomers), or which do not behave as image and mirror image (diastereomers). The invention relates both to the enantiomers or diastereomers and to their respective mixtures. These mixtures of the enantiomers and diastereomers can be separated in a known manner into the stereoisomerically uniform constituents.

Preferred compounds are those in which the hydroxy group forms the anti isomer (Ib).

The compounds of the general formula (I) according to the invention are obtained by oxidizing compounds of the general formula (II) 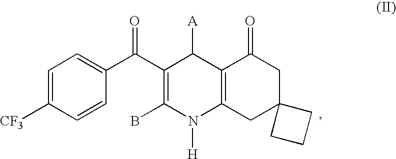
in which
A and B have the meanings indicated above,
   firstly to give the compounds of the general formula (III) 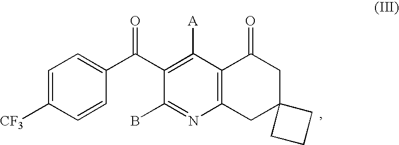
   in which
A and B have the meanings indicated above,
   reacting these in a next step by means of an asymmetric reduction to give the compounds of the general formula (IV) 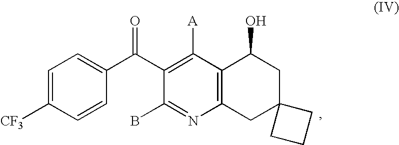
   in which
A and B have the meanings indicated above,
   then converting these
[A] by the introduction of a hydroxy protective group into the compounds of the general formula (V) 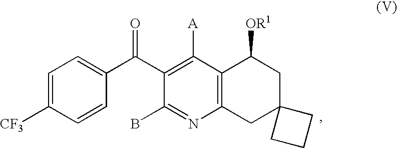
   in which
   R¹ represents a hydroxy protective group, preferably a radical of the formula —SiR²R³R⁴,
      in which
      R², R³ and R⁴ are identical or different and denote C₁-C₄-alkyl,
         preparing from these in a subsequent step by diastereoselective reduction the compounds of the general formula (VI) 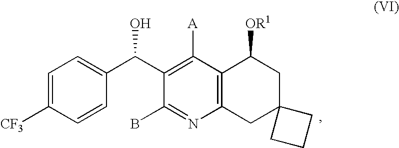
   in which
   R¹, A and B have the meanings indicated above,
   and subsequently cleaving the hydroxy protective group according to customary methods,
      or
[B] directly reducing the compounds of the formula (IV).

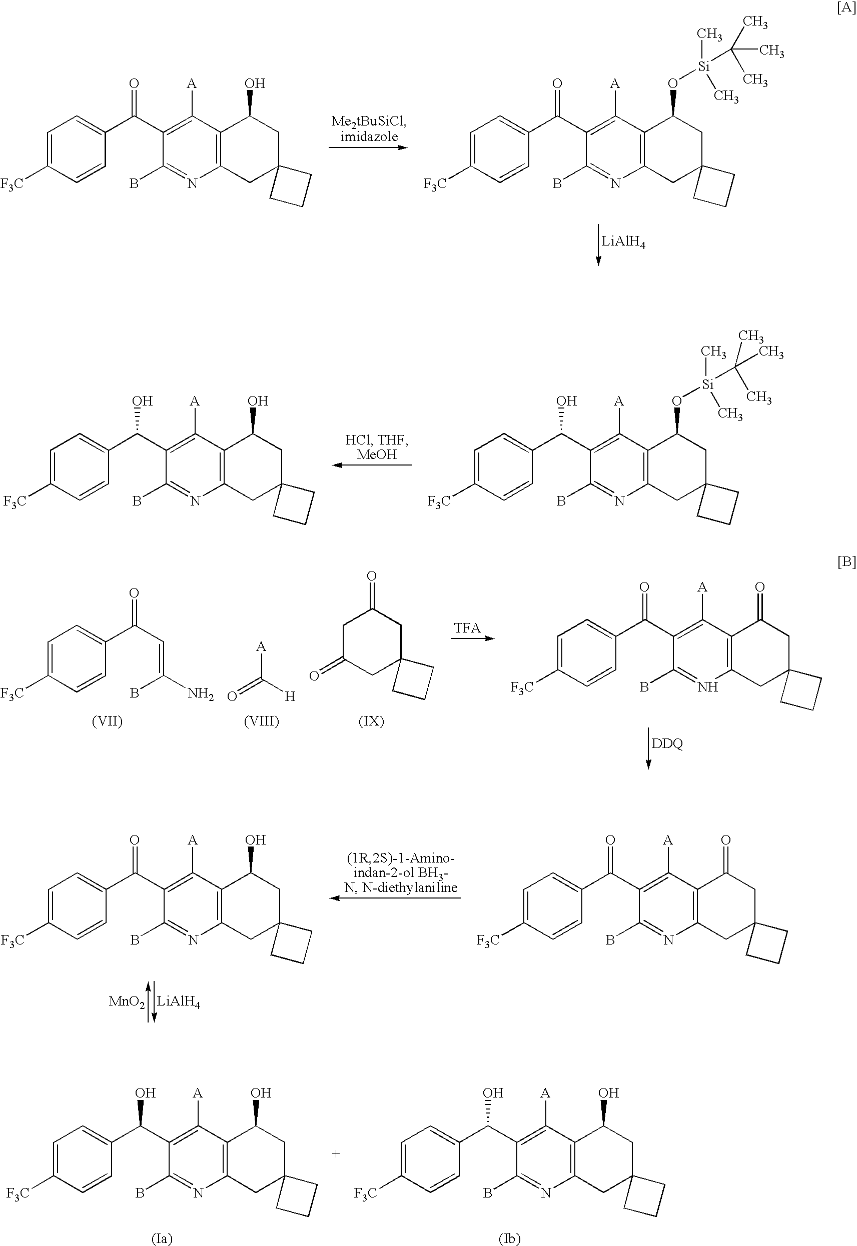

Suitable solvents for all processes are ethers such as diethyl ether, dioxane, tetrahydrofuran, glycol dimethyl ether, or hydrocarbons such as benzene, toluene, xylene, hexane, cyclohexane or petroleum fractions, or halogenohydrocarbons such as dichloromethane, trichloromethane, tetrachloromethane, dichloroethylene, trichloroethylene or chlorobenzene, or ethyl acetate, or triethylamine, pyridine, dimethyl sulphoxide, dimethylformamide, hexamethylphosphoramide, acetonitrile, acetone or nitromethane. It is likewise possible to use mixtures of the solvents mentioned. Dichloromethane is preferred.

Suitable bases for the individual steps are the customary strongly basic compounds. These preferably include organolithium compounds such as, for example, N-butyllithium, sec-butyllithium, tert-butyllithium or phenyllithium, or amides such as, for example, lithium diisopropylamide, sodium amide or potassium amide, or lithium hexamethylsilylamide, or alkali metal hydrides such as sodium hydride or potassium hydride. N-Butyllithium, sodium hydride or lithium diisopropylamide is particularly preferably employed.

The reductions are in general carried out using reducing agents, preferably using those which are suitable for the reduction of ketones to hydroxy compounds. Reduction using metal hydrides or complex metal hydrides in inert solvents, optionally in the presence of a trialkylborane, is particularly suitable here. Preferably, the reduction is carried out using complex metal hydrides such as, for example, lithium borohydride, sodium borohydride, potassium borohydride, zinc borohydride, lithium trialkylborohydride, diisobutylaluminium hydride or lithium aluminium hydride. The reduction is very particularly preferably carried out using diisobutylaluminium hydride or sodium borohydride.

The reducing agent is in general employed in an amount from 1 mol to 6 mol, preferably from 1 mol to 4 mol, relative to 1 mol of the compounds to be reduced.

The reduction in general proceeds in a temperature range from −78° C. to +50° C., preferably from −78° C. to 0° C. in the case of DIBAH, 0° C. to room temperature in the case of NaBH₄, particularly preferably at −78° C., in each case depending on the choice of the reducing agent and solvent.

The reduction in general proceeds at normal pressure, but it is also possible to work at elevated or reduced pressure.

The hydrogenation is carried out according to customary methods using hydrogen in the presence of noble metal catalysts, such as, for example, Pd/C, Pt/C or Raney nickel in one of the abovementioned solvents, preferably in alcohols such as, for example, methanol, ethanol or propanol, in a temperature range from −20° C. to +100° C., preferably from 0° C. to +50° C., at normal pressure or elevated pressure.

The removal of the protective group is in general carried out in one of the abovementioned alcohols and THF, preferably methanol/THF in the presence of hydrochloric acid in a temperature range from 0° C. to 50° C., preferably at room temperature, and normal pressure. In particular cases, the cleavage of the protective group using tetrabutylammonium fluoride (TBAF) in THF is preferred.

Hydroxy protective group in the context of the definition indicated above in general represents a protective group from the series: trimethylsilyl, triisopropylsilyl, tert-butyl-dimethylsilyl, benzyl, benzyloxycarbonyl, 2-nitrobenzyl, 4-nitrobenzyl, tert-butyloxycarbonyl, allyloxycarbonyl, 4-methoxybenzyl, 4-methoxybenzyloxycarbonyl, tetrahydropyranyl, formyl, acetyl, trichloroacetyl, 2.2.2-trichloroethoxycarbonyl, methoxyethoxymethyl, [2-(trimethylsilyl)ethoxy]methyl, benzoyl, 4-methylbenzoyl, 4-nitrobenzoyl, 4-fluorobenzoyl, 4-chlorobenzoyl or 4-methoxybenzoyl. Tetrahydropyranyl, tert-butyldimethylsilyl and triisopropylsilyl are preferred. tert-Butyldimethylsilyl is particularly preferred.

Suitable solvents for the individual steps are ethers such as diethyl ether, dioxane, tetrahydrofuran, glycol dimethyl ether, diisopropyl ether or hydrocarbons such as benzene, toluene, xylene, hexane, cyclohexane or petroleum fractions, or halogenohydrocarbons such as dichloromethane, trichloromethane, tetrachloromethane, dichloroethylene, trichloroethylene or chlorobenzene. It is likewise possible to use mixtures of the solvents mentioned.

Suitable oxidizing agents for the preparation of the compounds of the general formula (III) are, for example, nitric acid, cerium(IV) ammonium nitrate, 2,3-dichloro-5,6-dicyanobenzoquinone, pyridinium chlorochromate (PCC), pyridinium chlorochromate on basic alumina, osmium tetroxide and manganese dioxide. Manganese dioxide and nitric acid are preferred.

The oxidation is carried out in one of the abovementioned chlorinated hydrocarbons and water. Dichloromethane and water are preferred.

The oxidizing agent is employed in an amount from 1 mol to 10 mol, preferably from 2 mol to 5 mol, relative to 1 mol of the compounds of the general formula (II).

The oxidation in general proceeds at a temperature from −50° C. to +100° C., preferably from 0° C. to room temperature.

The oxidation in general proceeds at normal pressure. However, it is also possible to carry out the oxidation at elevated or reduced pressure.

The asymmetric reduction to give the compounds of the general formula (IV) is in general carried out in one of the abovementioned ethers or toluene, preferably tetrahydrofuran and toluene.

The reduction is in general carried out using enantiomerically pure 1R,2S-amino-indanol and borane complexes such as BH₃×THF, BH₃×DMS and BH₃×(C₂H₅)₂NC₆H₅. The system boranediethylaniline/1R,2S-aminoindanol is preferred.

The reducing agent is in general employed in an amount from 1 mol to 6 mol, preferably from 1 mol to 4 mol, relative to 1 mol of the compounds to be reduced.

The reduction in general proceeds at a temperature from −78° C. to +50° C., preferably from 0° C. to 30° C.

The reduction in general proceeds at normal pressure, but it is also possible to work at elevated or reduced pressure.

The introduction of the hydroxy protective group is carried out in one of the abovementioned hydrocarbons, dimethylformamide or THF, preferably in toluene in the presence of lutidine in a temperature range from −20° C. to +50° C., preferably from −5° C. to room temperature and normal pressure.

Reagents for the introduction of the silyl protective group are in general tert-butyldimethylsilyl chloride or tert-butyldimethylsilyl trifluoromethanesulphonate. tert-Butyldimethylsilyl trifluoromethanesulphonate is preferred.

The reduction for the preparation of the compounds of the general formula (VI) is in general carried out using customary reducing agents, preferably those which are suitable for the reduction of ketones to hydroxy compounds. Reduction using metal hydrides or complex metal hydrides in inert solvents, optionally in the presence of a trialkylborane, is particularly suitable here. Preferably, the reduction is carried out using complex metal hydrides such as, for example, lithium borohydride, sodium borohydride, potassium borohydride, zinc borohydride, lithium trialkylborohydride, diisobutylaluminium hydride, sodium bis-(2-methoxyethoxy)-dihydroaluminate or lithium aluminium hydride. The reduction is very particularly preferably carried out using sodium bis-(2-methoxyethoxy)-dihydroaluminate.

The reducing agent is in general employed in an amount from 1 mol to 6 mol, preferably from 1 mol to 3 mol, relative to 1 mol of the compounds to be reduced.

The reduction in general proceeds at a temperature from −20° C. to +110° C., preferably from 0° C. to room temperature.

The reduction in general proceeds at normal pressure, but it is also possible to work at elevated or reduced pressure.

In the reduction to give the compounds of the general formula (VI), small amounts of the wrong diastereomer remain in the mother liquor. These residues can be reoxidized using customary oxidizing agents such as, for example, pyridinium chlorochromate (PCC) or activated manganese dioxide, in particular using activated manganese dioxide, to give protected (V) and can thus be added to the synthesis cycle without loss of yield.

The compounds of the general formula (II) can be prepared by reacting compounds of the general formulae (XVa), (XVIII) and (XIX) 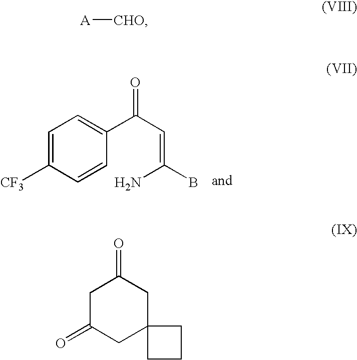
in which
A and B have the meaning indicated above,
   with an acid.

Suitable solvents for the preparation of the compounds of the general formula (II) are the abovementioned ethers or alcohols. Diisopropyl ether is preferred.

Suitable acids for the preparation of the compounds of the general formula (II) are in general organic carboxylic acids and inorganic acids, such as, for example, oxalic acid, maleic acid, phosphoric acid, fumaric acid and trifluoroacetic acid. Trifluoroacetic acid is preferred.

The acid is in general employed in an amount from 0.1 mol to 5 mol, preferably 1 mol, relative to 1 mol of the compounds of the general formula (IX).

The reaction is in general carried out at normal pressure. However, it is also possible to carry out the reaction at elevated or reduced pressure.

The reaction is in general carried out at the reflux temperature of the respective solvent.

The compounds of the general formulae (VII), (VIII) and (IX) are known per se or can be prepared according to customary methods.

The compounds of the general formula (I) according to the invention have valuable pharmacological properties and can be used for the prevention and treatment of diseases. In particular, the compounds according to the invention are highly active inhibitors of the cholesterol ester transfer protein (CETP) and stimulate reverse cholesterol transport. The active compounds according to the invention cause a lowering of the LDL cholesterol level (low density lipoprotein) in the blood together with a simultaneous increase in the HDL cholesterol level (high density lipoprotein). They can therefore be employed for the treatment and prevention of hypolipoproteinaemia, dyslipidaemias, hypertriglyceridaemias, hyperlipidaemias or arteriosclerosis. The active compounds according to the invention can moreover also be employed for the treatment and prevention of adiposity and obesity. The active compounds according to the invention are furthermore suitable for the treatment and prevention of stroke and of Alzheimer's disease.

The active compounds according to the invention open up a further treatment alternative and represent an enrichment of pharmacy. In comparison to the known and previously employed preparations, the compounds according to the invention show an improved spectrum of action. They are preferably distinguished by great specificity, good tolerability and lower side-effects, in particular in the cardiovascular area. An advantage of the compounds according to the invention, in addition to their high activity, is in particular reduced deposition behaviour in the fatty tissue.

The pharmacological action can be detected by means of known CETP inhibition tests.

The new active compounds can be administered on their own and, if needed, also in combination with other active compounds, preferably from the group consisting of CETP inhibitors, antidiabetics, antioxidants, cytostatics, calcium antagonists, hypotensive agents, thyromimetics, inhibitors of HMG-CoA reductase, inhibitors of HMG-CoA reductase gene expression, squalene synthesis inhibitors, ACAT inhibitors, circulation-promoting agents, platelet aggregation inhibitors, anticoagulants, angiotensin II receptor antagonists, cholesterol absorption inhibitors, MTP inhibitors, aldose reductase inhibitors, fibrates, niacin, anorectics, lipase inhibitors and PPAR agonists.

The combination of the compounds of the general formula (I) according to the invention with a glucosidase and/or amylase inhibitor for the treatment of familial hyperlipidaemias, adiposity and diabetes mellitus is preferred. Glucosidase and/or amylase inhibitors in the context of the invention are, for example, acarbose, adiposine, voglibose, miglitol, emiglitate, MDL-25637, camiglibose (MDL-73945), tendamistate, AI-3688, trestatin, pradimicin-Q and salbostatin.

The combination of acarbose, miglitol, emiglitate or voglibose with one of the abovementioned compounds of the general formula (I) according to the invention is also preferred.

Combinations of the compounds according to the invention with cholesterol-lowering statins, HDL-raising principles, bile acid absorption blockers, cholesterol absorption blockers, vasoactive principles or ApoB-lowering principles in order to treat dyslipidaemias, combined hyperlipidaemias, hypercholesterolaemias or hypertriglyceridaemias are furthermore preferred.

The combinations mentioned can also be employed for the primary or secondary prevention of coronary heart diseases (e.g. myocardial infarct).

Statins in the context of the invention are, for example, lovastatin, simvastatin, pravastatin, fluvastatin, atorvastatin, rosuvastatin and cerivastatin. ApoB-lowering agents are, for example, MTP inhibitors, vasoactive principles can be, for example—but not exclusively—adhesion inhibitors, chemokine receptor antagonists, cell proliferation inhibitors or substances having dilatory activity.

The combination of statins or ApoB inhibitors with one of the abovementioned compounds of the general formula (I) according to the invention is preferred.

The active compounds can act systemically and/or locally. For this purpose, they can be administered in a suitable manner, such as, for example, orally, parenterally, pulmonarily, nasally, sublingually, lingually, buccally, rectally, transdermally, conjunctivally, optically or as an implant.

For this administration route, the active compound can be administered in suitable administration forms.

For oral administration, known administration forms delivering the active compound rapidly and/or in modified form, such as, for example, tablets (uncoated and coated tablets, e.g. tablets provided with enteric coatings or film-coated tablets), capsules, sugar-coated tablets, granules, pellets, powders, emulsions, suspensions and solutions, are suitable.

Parenteral administration can be carried out with avoidance of an absorption step (intravenous, intra-arterial, intracardiac, intraspinal or intralumbal) or with involvement of an absorption (intramuscular, subcutaneous, intracutaneous, percutaneous, or intraperitoneal). Suitable administration forms for parental administration are, inter alia, injection and infusion preparations in the form of solutions, suspensions, emulsions, lyophilysates and sterile powders.

For the other administration routes, for example, pharmaceutical forms for inhalation (inter alia powder inhalers, nebulizers), nasal drops/solutions, sprays; tablets or capsules to be administered lingually, sublingually or buccally or capsules, suppositories, aural and ophthalmic preparations, vaginal capsules, aqueous suspensions (lotions, shake mixtures), lipophilic suspensions, ointments, creams, milk, pastes, dusting powder or implants are suitable.

The new active compounds are used for the production of medicaments, in particular for the production of medicaments for the prevention and treatment of the abovementioned diseases.

Medicaments are prepared in a known manner by converting the compounds according to the invention into the customary formulations, such as tablets, coated tablets, pills, granules, aerosols, syrups, emulsions, suspensions and solutions. This is carried out using inert non-toxic, pharmaceutically suitable excipients. These include, inter alia, vehicles (e.g. microcrystalline cellulose), solvents (e.g. liquid polyethylene glycols), emulsifiers (e.g. sodium dodecyl sulphate), dispersing agents (e.g. polyvinylpyrrolidone), synthetic and natural biopolymers (e.g. albumin), stabilizers (e.g. antioxidants such as ascorbic acid), colourants (e.g. inorganic pigments such as iron oxides) or taste and/or odour corrigents. In this connection, the therapeutically active compound should in each case be present in a concentration of approximately 0.5 to 90% by weight of the total mixture, i.e. in amounts which are sufficient in order to achieve the dosage range indicated.

The formulations are prepared, for example, by extending the active compounds using solvents and/or vehicles, if appropriate using emulsifiers and/or dispersing agents, where, for example, if water is used as a diluent, organic solvents can optionally be used as auxiliary solvents.

Intravenous, parenteral, perlingual and in particular oral administration are preferred.

In the case of parenteral administration, solutions of the active compound using suitable liquid vehicles can be employed.

In general, it has proved advantageous in the case of intravenous administration to administer amounts of approximately 0.001 to 1 mg/kg, preferably approximately 0.01 to 0.5 mg/kg of body weight, to achieve efficaceous results, and in the case of oral administration the dose is approximately 0.01 to 100 mg/kg, preferably 0.01 to 20 mg/kg and very particularly preferably 0.1 to 10 mg/kg of body weight.

In spite of this, if appropriate it may be necessary to depart from the amounts mentioned, namely depending on the body weight or the type of administration route, on individual behaviour towards the medicament, the manner of its formulation and the time or interval at which administration takes place. Thus in some cases it may be sufficient to manage with less than the abovementioned minimum amount, while in other cases the upper limit mentioned has to be exceeded. In the case of the administration of relatively large amounts, it may be advisable to divide these into a number of individual doses over the course of the day.

The following examples serve to illustrate the invention. The invention is not thereby restricted to the examples.

### EXAMPLES

1. 1-Isopropyl-3-(4-trifluoromethylphenyl)-propane-1,3-dione

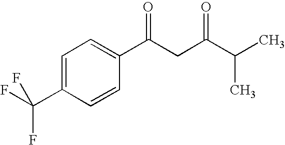

627.6 g (5.59 mol, 1.7 eq.) of potassium tert-butoxide are introduced into 3 1 of THF and 13.9 g (0.05 mol, 0.016 eq.) of 18-crown-6 ether are added. A solution of 619 g (3.29 mol, 1 eq.) of trifluoromethylacetophenone in 1.5 l of THF and a solution of 672 g (6.58 mol, 2 eq.) of methyl isobutyrate in 1.5 l of THF are then added dropwise simultaneously at RT from 2 dropping funnels within the course of 15 min. The mixture is then stirred under reflux for 4 hours. After cooling, 4 l of 10% hydrochloric acid are added dropwise at 0° C., the organic phase is separated off and the aqueous phase is extracted with 2 l of ethyl acetate. The organic phase is washed four times with 2 l of NaCl solution each time, dried over sodium sulphate, concentrated and the residue is distilled.

Yield: 618 g (69.8%)

¹H-NMR (CDCl₃, 300 MHz) δ=1.2 (d, 6H), 2.6 (sept, 1H), 6.2 (s, 1H), 7.7 (m, 2H), 8.0 (m, 2H), 16.1 (s, 1H) ppm.
2. 3-Amino-3-isopropyl-1-(4-trifluoromethylphenyl)-propenone

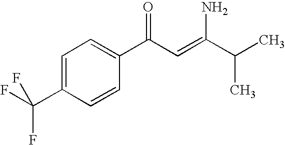

617 g (2.39 mol, 1 eq.) of the compound from Example 1 and 305.7 g (3.97 mol, 1.66 eq.) of ammonium acetate are dissolved in ethanol and stirred under reflux for 4 hours. The solution is then concentrated, washed with saturated sodium hydrogen-carbonate solution, dried over sodium sulphate and concentrated. The product is crystallized from cyclohexane.

Yield: 502 g (80.3%)

¹H-NMR (CDCl₃, 300 MHz) δ=1.2 (d, 3H), 2.5 (sept, 1H), 5.4 (br.s, 1H), 5.7 (s, 1H), 7.7 (m, 2H), 8.0 (m, 2H), 10.5 (br.s, 1H) ppm.
3. 1-Cyclopentyl-3-(4-trifluoromethylphenyl)-propane-1,3-dione

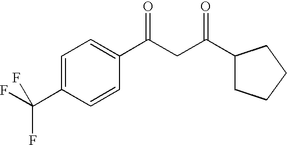

226.8 g (2.02 mol) of potassium tert-butoxide, 5.05 g (0.019 mol) of 18-crown-6 ether, 225 g (1.20 mol) of trifluoromethylacetophenone and 305.7 g (2.39 mol) of methyl cyclopentylcarboxylate are reacted analogously to the procedure of Example 1.

Yield: 256 g (75.3%)

¹H-NMR (CDCl₃, 200 MHz) δ=1.5-2.0 (compl. region, 8H), 2.9 (m, 1H), 6.2 (s, 1H), 7.7 (m, 2H), 8.0 (m, 2H), 16.1 (s, 1H) ppm.
4. 3-Amino-3-cyclopentyl-1-(4-trifluoromethylphenyl)-propenone

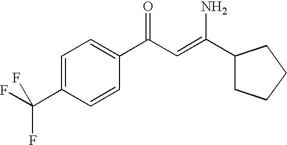

1622.6 g (5.7 mol) of the compound from Example 3 and 730 g (9.48 mmol) of ammonium acetate are reacted analogously to the procedure of Example 2.

Yield: 1028 g (63%)

¹H-NMR (CDCl₃, 200 MHz) δ=1.7 (m, 6H), 2.1 (m, 2H), 2.7 (m, 1H), 5.4 (br.s, 1H), 5.8 (s, 1H), 7.7 (m, 2H), 8.0 (m, 2H), 10.5 (br.s, 1H) ppm.
5. Cyclobutyl-dimedone (spiro[3,5]nonane-6,8-dione)

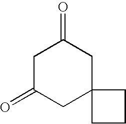

500 ml of 30% strength NaOMe in methanol are introduced and diluted with 640 ml of methanol. 359 g of dimethyl malonate are added to this at about 60° C. and the mixture is heated to reflux for 10 min. 300 g of cyclobutylidene-2-propanone are then added and the mixture is heated under reflux for 4 hours. For hydrolysis, 336 g of KOH dissolved in 1600 ml of water are added and the mixture is heated under reflux for 1 hour. It is then acidified with 20% strength hydrochloric acid and stirred at pH 3 to 5 until the end of the evolution of CO₂. After distillation of the methanol, the mixture is stirred with cooling to room temperature and the precipitated solid is isolated and washed until neutral and dried at 55° C. in vacuo.

Yield: 412 g corresponding to 99.4% of theory (NMR, DMSO, 1.7-1.95 ppm m (6H); 2.4 ppm s (4H), 5.2 ppm s (1H); 11.1 ppm br.s (—OH).
6. 2-Isopropyl-4-(4-fluorophenyl)-7-spirocyclobutyl-3-(4-trifluoromethylbenzoyl)-4,6,7,8-tetrahydro-1H-quinolin-5-one

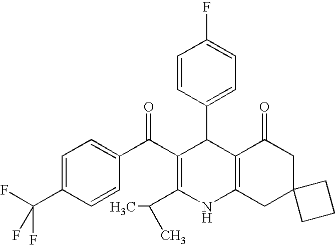

507 mg (1.97 mmol, 1.2 eq.) of the compound from Example 2 are introduced into 20 ml of diisopropyl ether and 0.253 ml (3.29 mmol, 2 eq.) of trifluoroacetic acid and 250 mg (1.64 mmol, 1 eq.) of spiro[3,5]nonane-6,8-dione are added. After stirring at room temperature for 10 min, 0.264 ml (2.46 mmol, 1.5 eq.) of 4-fluorobenzaldehyde is added and the mixture is heated under reflux for 18 h. After cooling, it is stirred in an ice bath for 15 min, and the precipitate obtained is filtered off with suction and washed with cold diisopropyl ether.

Yield: 640 mg (78.3%)

¹H-NMR (CDCl₃, 200 MHz) δ=1.1 (t, 3H), 1.2 (t, 3H), 1.7 (m, 2H), 1.9 (m, 4H), 2.4 (d, 1H), 2.7 (d, 1H), 2.6 (s, 2H), 3.1 (sept, 1H), 4.9 (s, 1H), 5.8 (s, 1H), 6.8 (m, 2H), 7.0 (m, 2H), 7.6 (m, 4H) ppm.
7. 2-Cyclopentyl-4-(4-fluorophenyl)-7-spirocyclobutyl-3-(4-trifluoromethylbenzoyl)-4,6,7,8-tetrahydro-1H-quinolin-5-one

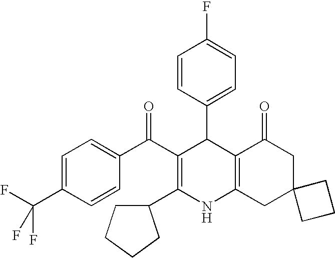

Analogously to the procedure of Example 6, 1.03 g (3.64 mmol) of the compound from Example 4, 678 mg (5.46 mmol) of 4-fluorbenzaldehyde and 834 mg (5.46 mmol) of spiro[3,5]nonane-6,8-dione are reacted.

Yield: 1.41 g (68%)

¹H-NMR (CDCl₃, 300 MHz) δ=1.38-2.03 (m, 14 H); 2.43 (d, 1H); 2.56 (d, 1H); 2.59 (m, 2H); 3.06 (m., 1H); 4.96 (s, 1H); 5.75 (s, 1H); 6.77-6.86 (m, 2H); 6.97-7.05 (m, 2H); 7.59-7.69 (m, 4 H) ppm.
8. 2-Isopropyl-4-phenyl-7-spirocyclobutyl-3-(4-trifluoromethylbenzoyl)-4,6,7,8-tetrahydro-1H-quinolin-5-one

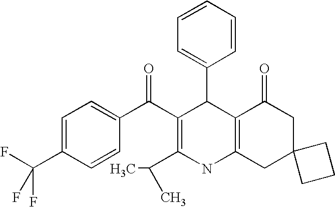

Analogously to the procedure of Example 6, 507 mg (1.97 mmol) of the compound from Example 2, 0.25 ml (2.46 mmol) of benzaldehyde and 250 mg (1.64 mmol, 1 eq.) of spiro[3,5]nonane-6,8-dione are reacted.

Yield: 272 mg (34.6%)

LC/MS (B) rt 4.82 min, MS (ES⁺): 480 [M+H]
9. 2-Cyclopentyl-4-phenyl-7-spirocyclobutyl-3-(4-trifluoromethylbenzoyl)-4,6,7,8-tetrahydro-1H-quinolin-5-one

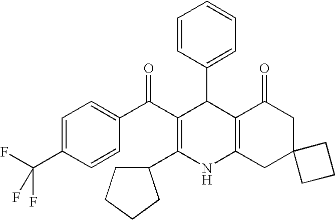

Analogously to the procedure of Example 6, 558 mg (1.97 mmol) of the compound from Example 4, 0.25 ml (2.46 mmol) of benzaldehyde and 250 mg (1.64 mmol, 1 eq.) of spiro[3,5]nonane-6,8-dione are reacted.

Crude yield: 193 mg (23%)

LC/MS (A) rt 3.5 min, MS (ESI): 506 [M+H]
10. 2-Isopropyl-4-(2-thienyl)-7-spirocyclobutyl-3-(4-trifluoromethylbenzoyl)-4,6,7,8-tetrahydro-1H-quinolin-5-one

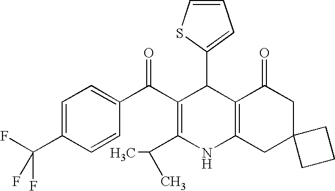

Analogously to the procedure of Example 6, 507 mg (1.97 mmol) of the compound from Example 2, 0.23 ml (2.46 mmol) of 2-thiophenecarbaldehyde and 250 mg (1.64 mmol, 1 eq.) of spiro[3,5]nonane-6,8-dione are reacted.

Crude yield: 450 mg (56.4%)
11. 2-Cyclopentyl-4-(3-thienyl)-7-spirocyclobutyl-3-(4-trifluoromethylbenzoyl)-4,6,7,8-tetrahydro-1H-quinolin-5-one

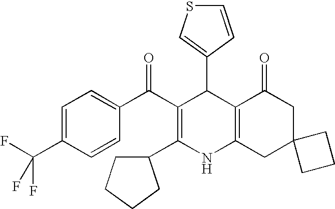

Analogously to the procedure of Example 6, 558 mg (1.97 mmol) of the compound from Example 4, 0.22 ml (2.46 mmol) of 3-thiophenecarbaldehyde and 250 mg (1.64 mmol, 1 eq.) of spiro[3,5]nonane-6,8-dione are reacted.

Crude yield: 261 mg (31%)

LC/MS (A) rt 3.5 min, MS (ESI): 512 [M+H]
12. 2-Isopropyl-4-(3-thienyl)-7-spirocyclobutyl-3-(4-trifluoromethylbenzoyl)-4,6,7,8-tetrahydro-1H-quinolin-5-one

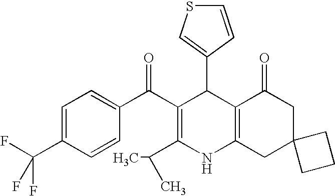

Analogously to the procedure of Example 6, 568 mg (2.21 mmol) of the compound from Example 2, 0.24 ml (2.76 mmol) of 3-thiophenecarbaldehyde and 280 mg (1.84 mmol, 1 eq.) of spiro[3,5]nonane-6,8-dione are reacted.

Yield: 599 mg (67%)

¹H-NMR (CDCl₃, 200 MHz) δ=1.1 (t, 3H), 1.2 (t, 3H), 1.7 (m, 1H), 1.8 (m, 2H), 1.9 (m, 3H), 2.5 (d, 1H), 2.7 (d, 1H), 2.6 (s, 2H), 3.2 (sept, 1H), 5.1 (s, 1H), 5.9 (s, 1H), 6.8 (m, 2H), 7.1 (m, 1H), 7.7 (m, 4H) ppm.
13. 2-Cyclopentyl-4-(2-thienyl)-7-spirocyclobutyl-3-(4-trifluoromethylbenzoyl)-4,6,7,8-tetrahydro-1H-quinolin-5-one

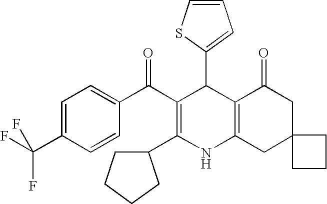

Analogously to the procedure of Example 6, 558 mg (1.97 mmol) of the compound from Example 4, 276 mg (2.46 mmol) of 2-thiophenecarbaldehyde and 250 mg (1.64 mmol, 1 eq.) of spiro[3,5]nonane-6,8-dione are reacted.

Crude yield: 500 mg (60%)
14. 2-Isopropyl-4-cyclohexyl-7-spirocyclobutyl-3-(4-trifluoromethylbenzoyl)-4,6,7,8-tetrahydro-1H-quinolin-5-one

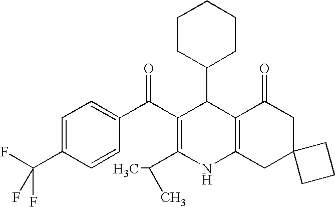

Analogously to the procedure of Example 6, 1.038 g (4.04 mmol) of the compound from Example 2, 0.611 ml (5.05 mmol) of cyclohexanecarbaldehyde and 571 mg (3.36 mmol, 1 eq.) of spiro[3,5]nonane-6,8-dione are reacted.

Yield: 726 mg (44.4%)

¹H-NMR (CDCl₃, 200 MHz) δ=0.9 (m, 6H), 1.1 (d, 3H), 1.3 (d, 3H), 1.5 (m, 4H), 2.0 (m, 7H9, 2.5 (d, 1H), 2.6 (s, 2H), 2.7 (d, 1H), 3.5 (sept, 1H), 3.7 (d, 1H), 5.9 (s, 1H), 7.7 (m, 2H), 7.8 (m, 2H) ppm.
15. 2-Cyclopentyl-4-cyclohexyl-7-spirocyclobutyl-3-(4-trifluoromethylbenzoyl)-4,6,7,8-tetrahydro-1H-quinolin-5-one

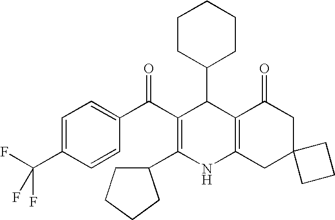

Analogously to the procedure of Example 6, 893 mg (3.15 mmol) of the compound from Example 4, 0.48 ml (3.94 mmol) of cyclohexanecarbaldehyde and 398 mg (2.62 mmol, 1 eq.) of spiro[3,5]nonane-6,8-dione are reacted.

Yield: 350 mg (26%)

¹H-NMR (CDCl₃, 200 MHz) δ=1.0 (m, 6H), 1.3 (m, 1H), 1.6 (m, 6H), 1.7 (m, 6H), 1.9 (m, 6H), 2.2 (m, 1H), 2.4 (d, 1H), 2.6 (s, 2H), 2.7 (d, 1H), 3.7 (d, 1H), 5.9 (s, 1H), 7.6 (m, 2H), 7.8 (m, 2H) ppm.
16. 2-Isopropyl-4-cyclopentyl-7-spirocyclobutyl-3-(4-trifluoromethylbenzoyl)-4,6,7,8-tetrahydro-1H-quinolin-5-one

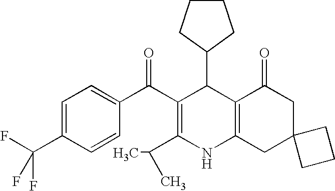

Analogously to the procedure of Example 6, 1.014 g (3.94 mmol) of the compound from Example 2, 0.689 ml (6.57 mmol) of cyclopentanecarbaldehyde and 499 mg (3.28 mmol, 1 eq.) of spiro[3,5]nonane-6,8-dione are reacted.

Yield: 299 mg (19%)

¹H-NMR (CDCl₃, 200 MHz) δ=0.9 (m, 2H), 1.1 (t, 3H), 1.3 (t, 3H), 1.3-1.6 (m, 6H), 2.0 (m, 6H), 2.4 (d, 1H), 2.6 (s, 2H), 2.7 (d, 1H), 3.5 (sept, 1H), 3.8 (d, 1H), 7.6 (m, 2H), 7.8 (m, 2H) ppm.
17. 2,4-Dicyclopentyl-7-spirocyclobutyl-3-(4-trifluoromethylbenzoyl)-4,6,7,8-tetrahydro-1H-quinolin-5-one

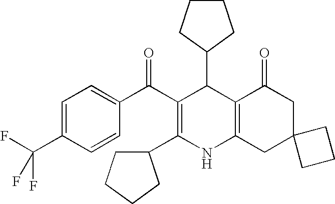

Analogously to the procedure of Example 6, 1.116 g (3.94 mmol) of the compound from Example 4, 0.689 ml (6.57 mmol) of cyclopentanecarbaldehyde and 499 mg (3.28 mmol, 1 eq.) of spiro[3,5]nonane-6,8-dione are reacted.

Crude yield: 300 mg (18.3%)
18. 2-Cyclopentyl-4-cyclobutyl-7-spirocyclobutyl-3-(4-trifluoromethylbenzoyl)-4,6,7,8-tetrahydro-1H-quinolin-5-one

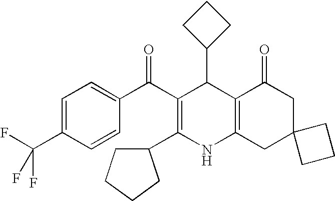

Analogously to the procedure of Example 6, 1.116 g (3.94 mmol) of the compound from Example 4, 0.591 ml (6.57 mmol) of cyclobutanecarbaldehyde and 499 mg (3.28 mmol, 1 eq.) of spiro[3,5]nonane-6,8-dione are reacted.

Crude yield: 1.11 g (70%)

LC/MS (A) rt 3.6 min, MS (ESI): 484 [M+H]
19. 2-Cyclopentyl-4-isopropyl-7-spirocyclobutyl-3-(4-trifluoromethylbenzoyl)-4,6,7,8-tetrahydro-1H-quinolin-5-one

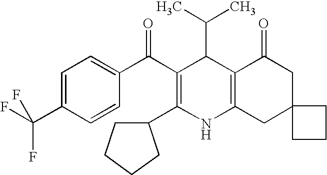

Analogously to the procedure of Example 6, 1.116 g (3.94 mmol) of the compound from Example 4, 2.369 g (32.85 mmol, 10 eq.) of 2-methylpropionaldehyde and 499 mg (3.28 mmol, 1 eq.) of spiro[3,5]nonane-6,8-dione are reacted.

Yield: 202.5 mg (13.1%)

LC/MS (A) rt 3.69 min, MS (ESI): 472 [M+H]
20. 2-Cyclopentyl-4-(1-propyl)-7-spirocyclobutyl-3-(4-trifluoromethylbenzoyl)-4,6,7,8-tetrahydro-1H-quinolin-5-one

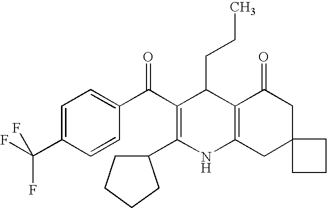

Analogously to the procedure of Example 6, 1.116 g (3.94 mmol) of the compound from Example 4, 2.96 ml (32.85 mmol, 10 eq.) of butanal and 499 mg (3.28 mmol, 1 eq.) of spiro[3,5]nonane-6,8-dione are reacted.

Yield: 192 mg (12.4%)

LC/MS (A) rt 3.71 min, MS (ESI): 472 [M+H]
21. 2-Isopropyl-4-(4-fluorophenyl)-7-spirocyclobutyl-3-(4-trifluoromethylbenzoyl)-7,8-dihydro-6H-quinolin-5-one

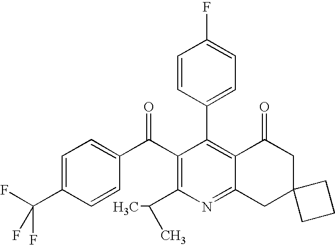

635 mg (1.28 mmol, 1 eq.) of the compound from Example 6 are dissolved in 20 ml of dichloromethane and stirred at room temperature with 318.7 mg (1.40 mmol, 1.1 eq.) of 2,3-dichloro-5,6-dicyano-1,4-benzoquinone (DDQ) for 1 h. The mixture is concentrated on a rotary evaporator and the product is isolated by chromatography (silica gel, elution with cyclohexane/ethyl acetate 20:1-10:1).

Yield: 573 mg (90.6%)

¹H-NMR (CDCl₃, 200 MHz) δ=1.2 (tr, 6H), 2.0 (m, 6H), 2.7 (s, 2H), 2.8 (sept, 1H), 3.4 (s, 2H), 6.5-7.0 (br. m, 4H), 7.6 (m, 4H) ppm.
22. 2-Cyclopentyl-4-(4-fluorophenyl)-7-spirocyclobutyl-3-(4-trifluoromethylbenzoyl)-7,8-dihydro-6H-quinolin-5-one

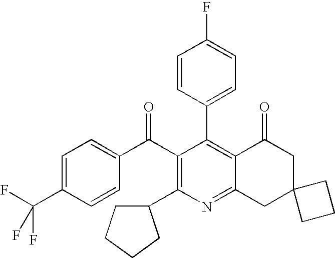

10 g (104 mmol) of manganese dioxide (Merck No. 805958—active, precipitated, about 90%) are added at room temperature to a solution of 1.375 g (2.43 mmol) of the compound from Example 7 in dichloromethane (30 ml). After stirring at room temperature for 1 h, the mixture is filtered through kieselguhr and a layer of sea sand and washed intensively with dichloromethane. The filtrate is concentrated in vacuo and the residue is taken up using a mixture of EA/PE 1:7 with addition of dichloromethane and purified by flash chromatography on silica gel using EA/PE 1:7. After removing the solvents, a yellowish white, crystalline solid is isolated.

Yield: 1.05 g (83%)

MS (ESI): 522 (M+H)

¹H-NMR (CDCl₃, 400 MHz) δ=1.5-2.1 (m, 14 H); 2.72 (s, 2H); 2.85 (m., 1H); 3.37 (s, 2H); 6.55-7.13 (br. m, 4H); 7.55-7.62 (m, 4H) ppm.
23. 2-Isopropyl-4-phenyl-7-spirocyclobutyl-3-(4-trifluoromethylbenzoyl)-7,8-dihydro-6H-quinolin-5-one

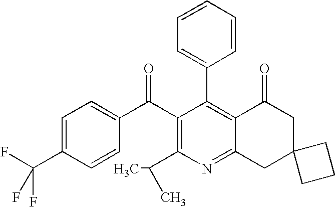

272 mg (0.57 mmol) of Example 8 are reacted analogously to the procedure of the compound from Example 21.

Yield: 262 mg (96.8%)

¹H-NMR (CDCl₃, 200 MHz) δ=1.2 (tr, 6H), 2.0 (m, 6H), 2.7 (s, 2H), 2.8 (sept., 1H), 3.4 (s, 2H), 6.8-7.2 (br. m, 4H), 7.6 (m, 4H) ppm.
24. 2-Cyclopentyl-4-phenyl-7-spirocyclobutyl-3-(4-trifluoromethylbenzoyl)-7,8-dihydro-6H-quinolin-5-one

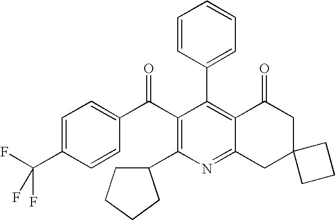

190 mg (0.38 mmol) of Example 9 are reacted analogously to the procedure of the compound from Example 21.

Yield: 20 mg (10.6%)

¹H-NMR (CDCl₃, 200 MHz) δ=1.8-2.1 (m, 12H), 2.7 (s, 2H), 2.9 (m, 1H), 3.4 (s, 2H), 6.7-7.1 (br. m, 4H), 7.6 (m, 4H) ppm.
25. 2-Isopropyl-4-(3-thienyl)-7-spirocyclobutyl-3-(4-trifluoromethylbenzoyl)-7,8-dihydro-6H-quinolin-5-one

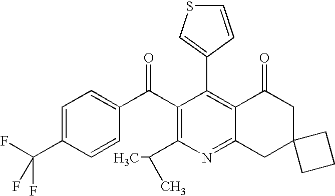

596 mg (1.23 mmol) of Example 12 are reacted analogously to the procedure of the compound from Example 21.

Yield: 553 mg (93.2%)

¹H-NMR (CDCl₃, 200 MHz) δ=1.2 (m, 6H), 2.0 (m, 6H), 2.7 (s, 2H), 2.8 (sept., 1H), 3.4 (s, 2H), 6.6 (m, 1H), 6.8 (m, 1H), 7.0 (m, 1H), 7.6 (m, 4H) ppm.
26. 2-Cyclopentyl-4-(3-thienyl)-7-spirocyclobutyl-3-(4-trifluoromethylbenzoyl)-7,8-dihydro-6H-quinolin-5-one

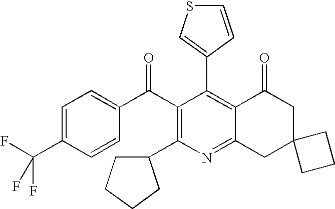

220 mg (0.43 mmol) of Example 11 are reacted analogously to the procedure of the compound from Example 21.

Yield: 180 mg (82.1%)

¹H-NMR (CDCl₃, 200 MHz) δ=1.8-2.1 (br. m, 12H), 2.7 (s, 2H), 2.9 (m, 1H), 3.3 (s, 2H), 6.6 (m, 1H), 6.8 (m, 1H), 7.0 (m, 1H), 7.6 (m, 4H) ppm.
27. 2-Isopropyl-4-(2-thienyl)-7-spirocyclobutyl-3-(4-trifluoromethylbenzoyl)-7,8-dihydro-6H-quinolin-5-one

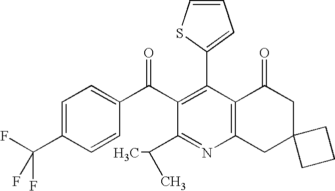

450 mg (0.93 mmol) of Example 10 are reacted analogously to the procedure of the compound from Example 21.

Yield: 400 mg (89.3%)

¹H-NMR (CDCl₃, 300 MHz) δ=1.2 (m, 6H), 2.0 (m, 6H), 2.7 (s, 2H), 2.8 (sept, 1H), 3.4 (s, 2H), 6.6 (m, 1H), 6.7 (m, 1H), 7.1 (m, 1H), 7.6 (m, 2H), 7.7 (m, 2H) ppm.
28. 2-Cyclopentyl-4-(2-thienyl)-7-spirocyclobutyl-3-(4-trifluoromethylbenzoyl)-7,8-dihydro-6H-quinolin-5-one

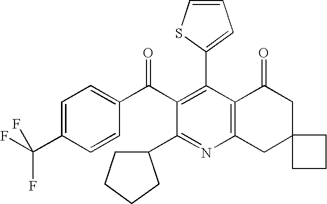

500 mg (0.98 mmol) of Example 13 are reacted analogously to the procedure of the compound from Example 21.

Yield 100 mg (20.1%)

¹H-NMR (CDCl₃, 200 MHz) δ=1.9-2.1 (m, 12H), 2.8 (s, 2H), 2.9 (m, 1H), 3.4 (s, 2H), 6.6 (m, 1H), 6.7 (m, 1H), 7.1 (m, 1H), 7.6 (m, 2H), 7.7 (m, 2H) ppm.
29. 2-Isopropyl-4-cyclohexyl-7-spirocyclobutyl-3-(4-trifluoromethylbenzoyl)-7,8-dihydro-6H-quinolin-5-one

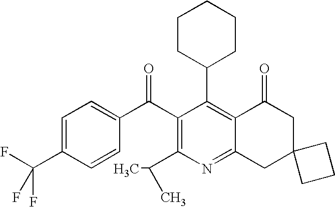

417 mg (0.86 mmol) of Example 14 are reacted analogously to the procedure of the compound from Example 21.

Yield: 399 mg (96%)

¹H-NMR (CDCl₃, 300 MHz) δ=1.0 (t, 3H), 1.1 (t, 3H), 1.4 (m, 1H), 1.5-1.7 (m, 8H), 1.8 (m, 1H), 2.0 (m, 6H), 2.6 (sept, 1H), 2.8 (s, 2H), 3.2 (m, 1H), 3.3 (s, 2H), 7.7 (m, 2H), 8.0 (m, 2H) ppm.
30. 2-Cyclopentyl-4-cyclohexyl-7-spirocyclobutyl-3-(4-trifluoromethylbenzoyl)-7,8-dihydro-6H-quinolin-5-one

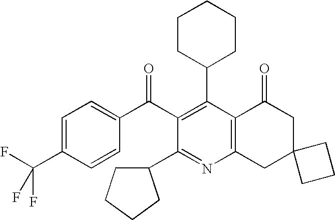

320 mg (0.63 mmol) of Example 15 are reacted analogously to the procedure of the compound from Example 21.

Yield: 300 mg (94%)

¹H-NMR (CDCl₃, 200 MHz) δ=1.1 (m, 2H), 1.4-1.6 (m, 10H), 1.8 (m, 6H), 2.0 (m, 6H), 2.6 (m, 1H), 2.8 (s, 2H), 3.2 (m, 1H), 3.3 (s, 2H), 7.7 (m, 2H), 8.0 (m, 2H) ppm.
31. 2-Isopropyl-4-cyclopentyl-7-spirocyclobutyl-3-(4-trifluoromethylbenzoyl)-7,8dihydro-6H-quinolin-5-one

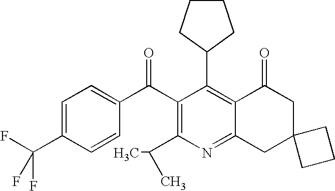

295 mg (0.63 mmol) of Example 16 are reacted analogously to the procedure of the compound from Example 21.

Yield: 290 mg (98.6%)

¹H-NMR (CDCl₃, 300 MHz) δ=1.1 (t, 3H), 1.2 (t, 3H), 1.4 (m, 3H), 1.7 (m, 1H), 1.8-2.1 (m, 10H), 2.6 (sept, 1H), 2.8 (s, 2H), 3.0 (m, 1H), 3.3 (s, 2H), 7.7 (m, 2H), 7.9 (m, 2H) ppm.
32. 2,4-Dicyclopentyl-7-spirocyclobutyl-3-(4-trifluoromethylbenzoyl)-7,8-dihydro-6H-quinolin-5-one

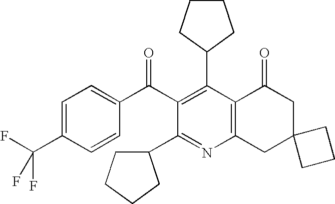

300 mg (0.60 mmol) of Example 17 are reacted analogously to the procedure of the compound from Example 21.

Yield: 200 mg (97.2%) LC/MS (A) rt 5.27 min, MS (ESI): 496 [M+H]
33. 2-Cyclopentyl-4-cyclobutyl-7-spirocyclobutyl-3-(4-trifluoromethylbenzoyl)-7,8-dihydro-6H-quinolin-5-one

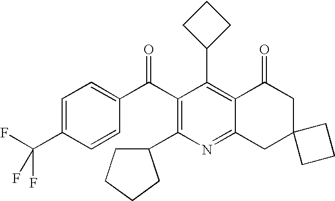

1.1 g (2.27 mmol) of Example 18 are reacted analogously to the procedure of the compound from Example 21.

Yield: 379 mg (35.6%)

¹H-NMR (CDCl₃, 200 MHz) δ=1.5 (m, 4H), 1.7-2.0 (m, 15H), 2.2 (m, 1H), 2.8 (m, 3H), 3.2 (s, 2H), 4.0 (pent, 1H), 7.7 (m, 2H), 7.9 (m, 2H) ppm.
34. 2-Cyclopentyl-4-isopropyl-7-spirocyclobutyl-3-(4-trifluoromethylbenzoyl)-7,8-dihydro-6H-quinolin-5-one

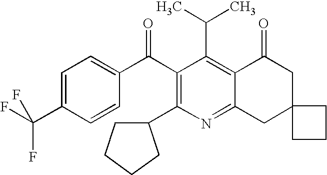

198 mg (0.42 mmol) of Example 19 are reacted analogously to the procedure of the compound from Example 21.

Yield: 132 mg (66.9%)

¹H-NMR (CDCl₃, 300 MHz) δ=1.1 (t, 3H), 1.2 (t, 3H), 1.5 (m, 2H), 1.8 (m, 4H), 2.0 (m, 8H), 2.6 (m, 1H), 2.8 (s, 2H), 3.2 (s, 2H), 3.4 (m, 1H), 7.7 (m, 2H), 7.9 (m, 2H) ppm.
35. 2-Cyclopentyl-4-(1-propyl)-7-spirocyclobutyl-3-(4-trifluoromethylbenzoyl)-7,8-dihydro-6H-quinolin-5-one

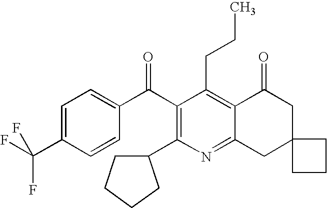

187 mg (0.40 mmol) of Example 20 are reacted analogously to the procedure of the compound from Example 21.

Yield: 121 mg (65%)

¹H-NMR (CDCl₃, 300 MHz) δ=0.8 (t, 3H), 1.3-1.6 (m, 4H), 1.8-2.1 (m, 12H), 2.3 (m, 1H), 2.7 (m, 1H), 2.8 (s, 2H), 3.2 (m, 1H), 3.3 (s, 2H), 7.7 (m, 2H), 7.9 (m, 2H) ppm.
36. [(5S)-2-Isopropyl-4-(4-fluorophenyl)-5-hydroxy-7-spirocyclobutyl-5,6,7,8-tetrahydroquinolin-3-yl]-(4-trifluoromethylphenyl)-methanone

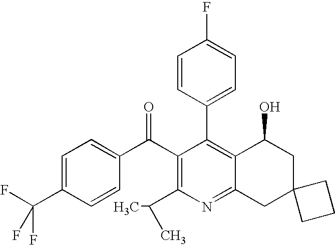

25.5 mg (0.17 mmol, 0.15 eq.) of (1R,2S)-1-aminoindan-2-ol are introduced into 10 ml THF and treated at room temperature with 743.5 mg (4.56 mmol, 4 eq.) of borane-N,N-diethylaniline complex. After the evolution of gas has ended, the mixture is cooled to 0° C. and 564.8 mg (1.14 mmol, 1 eq.) of Example 21, dissolved in 50 ml of tetrahydrofuran, are added. The mixture is allowed to come to room temperature over a number of hours. After reaction has taken place, the reaction mixture is treated with 1 ml of methanol, concentrated and the product is isolated by chromatography (silica gel, eluent cyclohexane/ethyl acetate mixtures).

Yield: quantitative

¹H-NMR (CDCl₃, 300 MHz) δ=1.2 (t, 6H), 2.0 (m, 6H), 2.1 (m, 1H), 2.3 (m, 1H), 2.8 (sept, 1H), 3.0 (d, 1H), 3.4 (d, 1H), 4.8 (br.s, 1H), 6.8 (m, 2H), 7.1 (m, 2H), 7.6 (m, 2H), 7.7 (m, 2H) ppm.
37. [(5S)-2-Cyclopentyl-4-(4-fluorophenyl)-5-hydroxy-7-spirocyclobutyl-5,6,7,8-tetrahydroquinolin-3-yl]-(4-trifluoromethylphenyl)-methanone

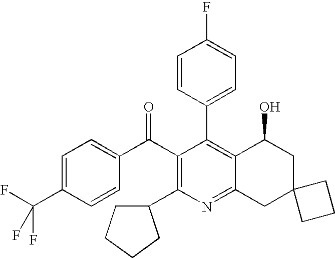

830 mg (1.59 mmol) of Example 22 are reacted analogously to the procedure of the compound from Example 36.

Yield: 783 mg (94%)

¹H-NMR (CDCl₃, 400 MHz) δ=1.33-1.45 (br. s, 1H); 1.46-1.6 (m, 2H); 1.7-2.15 (m, 13H); 2.20-2.30 (m, 1H); 2.82 (m, 1H); 2.97 (d, 1H); 3.41 (d, 1H); 4.75 (br. s; 1H); 6.75-7.20 (br. m, 4H); 7.55-7.62 (m, 2H); 7.62-7.70 (m, 2H) ppm.
38. [(5S)-2-Isopropyl-4-phenyl-5-hydroxy-7-spirocyclobutyl-5,6,7,8-tetrahydroquinolin-3-yl]-(4-trifluoromethylphenyl)-methanone

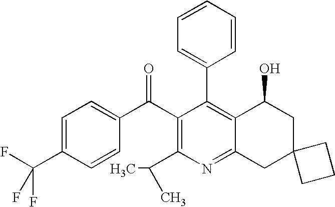

254 mg (0.53 mmol) of Example 23 are reacted analogously to the procedure of the compound from Example 36.

Yield: quantitative

¹H-NMR (CDCl₃, 300 MHz) δ=1.2 (t, 6H), 2.0 (m, 6H), 2.1 (m, 1H), 2.2 (m, 1H), 2.8 (sept, 1H), 3.0 (d, 1H), 3.4 (d, 1H), 4.9 (br.s., 1H), 7.1 (m, 4H), 7.6 (m, 2H), 7.7 (m, 2H) ppm.
39. [(5S)-2-Cyclopentyl-4-phenyl-5-hydroxy-7-spirocyclobutyl-5,6,7,8-tetrahydroquinolin-3-yl]-(4-trifluoromethylphenyl)-methanone

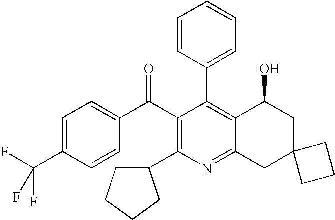

66 mg (0.13 mmol) of Example 24 are reacted analogously to the procedure of the compound from Example 36.

Yield: 62 mg (93.6%)

LC/MS (A) rt 3.68 min, MS (ESI): 506 [M+H]
40. [(5S)-2-Isopropyl-4-(3-thienyl)-5-hydroxy-7-spirocyclobutyl-5,6,7,8-tetrahydroquinolin-3-yl]-(4-trifluoromethylphenyl)-methanone

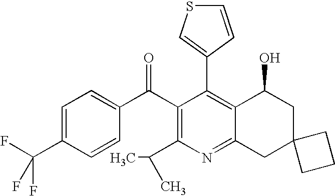

550 mg (1.14 mmol) of Example 25 are reacted analogously to the procedure of the compound from Example 36.

Yield: quantitative

¹H-NMR (CDCl₃, 300 MHz) δ=1.2 (m, 6H), 2.0 (m, 6H), 2.1 (m, 1H), 2.2 (m, 1H), 2.8 (sept, 1H), 3.0 (d, 1H), 3.4 (d, 1H), 4.9 (br.s, 1H), 6.8 (m, 1H), 7.1 (m, 1H), 7.2 (m, 1H), 7.6 (m, 2H), 7.7 (m, 2H) ppm.
41. [(5S)-2-Cyclopentyl-4-(3-thienyl)-5-hydroxy-7-spirocyclobutyl-5,6,7,8-tetrahydroquinolin-3-yl]-(4-trifluoromethylphenyl)-methanone

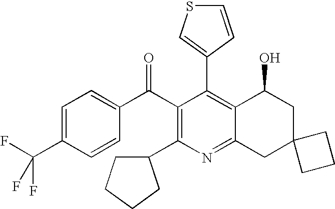

230 mg (0.45 mmol) of Example 26 are reacted analogously to the procedure of the compound from Example 36.

Yield: 200 mg (86.6%)

¹H-NMR (CDCl₃, 300 MHz) δ=1.8-2.0 (m, 14H), 2.1 (m, 1H), 2.2 (m, 1H), 2.9 (m, 1H), 3.0 (d, 1H), 3.4 (d, 1H), 4.9 (br.s, 1H), 6.8 (m, 1H), 7.0 (m, 1H), 7.1 (m, 1H), 7.6 (m, 2H), 7.7 (m, 2H) ppm.
42. [(5S)-2-Isopropyl-4-(2-thienyl)-5-hydroxy-7-spirocyclobutyl-5,6,7,8-tetrahydroquinolin-3-yl]-(4-trifluoromethylphenyl)-methanone

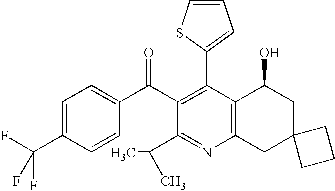

400 mg (0.83 mmol) of Example 27 are reacted analogously to the procedure of the compound from Example 36.

Yield: quantitative

¹H-NMR (CDCl₃, 300 MHz) δ=1.2 (m, 6H), 1.7 (br.s, 1H), 2.0 (m, 6H), 2.1 (m, 1H), 2.2 (m, 1H), 2.8 (sept, 1H), 3.0 (d, 1H), 3.4 (d, 1H), 5.0 (br.s, 1H), 6.9 (m, 2H), 7.2 (m, 1H), 7.6 (m, 2H), 7.7 (m, 2H) ppm.
43. [(5S)-2-Cyclopentyl-4-(2-thienyl)-5-hydroxy-7-spirocyclobutyl-5,6,7,8-tetrahydroquinolin-3-yl]-(4-trifluoromethylphenyl)-methanone

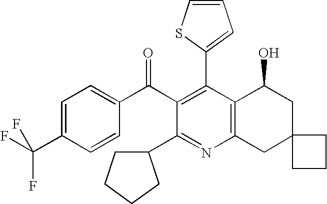

100 mg (0.20 mmol) of Example 28 are reacted analogously to the procedure of the compound from Example 36.

Yield: 87 mg (87%)

¹H-NMR (CDCl₃, 300 MHz) δ=1.8-2.0 (m, 14H), 2.1 (m, 1H), 2.3 (m, 1H), 2.8 (m, 1H), 3.0 (d, 1H), 3.4 (d, 1H), 5.0 (br.s, 1H), 6.8 (m, 2H), 7.2 (m, 1H), 7.6 (m, 2H), 7.7 (m, 2H) ppm.
44. [(5S)-2-Isopropyl-4-cyclohexyl-5-hydroxy-7-spirocyclobutyl-5,6,7,8-tetrahydroquinolin-3-yl]-(4-trifluoromethylphenyl)-methanone

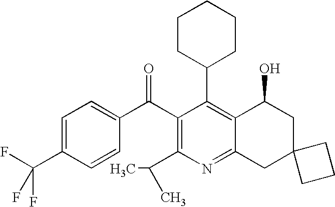

590 mg (1.22 mmol) of Example 29 are reacted analogously to the procedure of the compound from Example 36.

Yield: 526 mg (88.8%)

¹H-NMR (CDCl₃, 200 MHz) δ=1.1 (m, 8H), 1.4 (m, 1H), 1.5-1.7 (m, 6H), 1.9 (m, 6H), 2.2 (m, 3H), 2.5 (m, 1H), 2.9 (d, 1H), 3.2 (br.m, 1H), 3.4 (d/d, 1H), 5.2 (br.s, 1H), 7.7 (m, 2H), 7.9 (br.s, 2H) ppm.
45. [(5S)-2-Cyclopentyl-4-cyclohexyl-5-hydroxy-7-spirocyclobutyl-5,6,7,8-tetrahydroquinolin-3-yl]-(4-trifluoromethylphenyl)-methanone

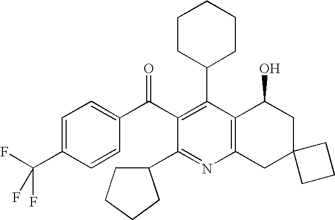

300 mg (0.59 mmol) of Example 30 are reacted analogously to the procedure of the compound from Example 36.

Yield 280 mg (93%)

¹H-NMR (DMSO-d₆, 200 MHz) δ=1.0-2.0 (compl. region., 25H), 2.1 (m, 1H), 2.3 (m, 1H), 2.8 (d/d, 1H), 3.2 (d, 1H), 5.0 (m, 1H), 7.9 (br.m, 4H) ppm.
46. [(5S)-2-Isopropyl-4-cyclopentyl-5-hydroxy-7-spirocyclobutyl-5,6,7,8-tetrahydroquinolin-3-yl]-(4-trifluoromethylphenyl)-methanone

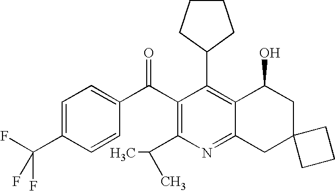

285 mg (0.61 mmol) of Example 31 are reacted analogously to the procedure of the compound from Example 36.

Yield: 263 mg (92%)

¹H-NMR (CDCl₃, 300 MHz) δ=1.2 (m, 6H), 1.5 (m, 4H), 1.7 (m, 2H), 2.0 (m, 6H), 2.1 (m, 1H), 2.3 (m.2H), 2.5 (sept, 1H), 2.9 (d, 1H), 3.3 (m, 1H), 3.5 (d/d, 1H), 5.1 (m, 1H), 7.7 (m, 2H), 7.9 (m, 2H) ppm.
47. [(5S)-2,4-Dicyclopentyl-5-hydroxy-7-spirocyclobutyl-5,6,7,8-tetrahydroquinolin-3-yl]-(4-trifluoromethylphenyl)-methanone

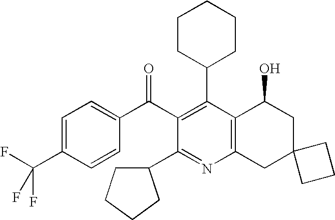

200 mg (0.4 mmol) of Example 32 are reacted analogously to the procedure of the compound from Example 36.

Yield: 175 mg (87.2%)

¹H-NMR (CDCl₃, 200 MHz) δ=1.4-2.1 (compl. region, 22H), 2.3 (m, 2H), 2.6 (m, 1H), 2.9 (d, 1H), 3.3 (m, 1H), 3.4 (m, 1H), 5.1 (m, 1H), 7.7 (m, 2H), 7.9 (m, 2H) ppm.
48. [(5S)-2-Cyclopentyl-4-cyclobutyl-5-hydroxy-7-spirocyclobutyl-5,6,7,8-tetrahydroquinolin-3-yl]-(4-trifluoromethylphenyl)-methanone

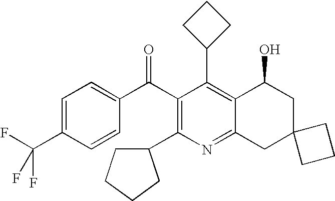

372 mg (0.77 mmol) of Example 33 are reacted analogously to the procedure of the compound from Example 36.

Yield: quantitative

¹H-NMR (CDCl₃, 200 MHz) δ=1.4 (m, 4H), 1.8 (m, 6H), 2.0 (m, 8H), 2.2 (m, 3H), 2.2 (m, 1H), 2.4 (m, 1H), 2.7 (m, 1H), 2.9 (d/d, 1H), 3.2 (d, 1H), 5.1 (d/tr, 1H), 7.7 (m, 2H), 8.0 (m, 2H) ppm.
49. [(5S)-2-Cyclopentyl-4-isopropyl-5-hydroxy-7-spirocyclobutyl-5,6,7,8-tetrahydroquinolin-3-yl]-(4-trifluoromethylphenyl)-methanone

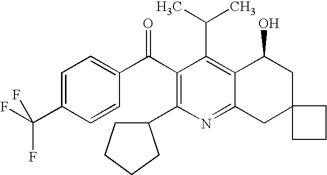

127 g (0.27 mmol) of Example 34 are reacted analogously to the procedure of the compound from Example 36.

Yield: 90 mg (70.7%)

¹H-NMR (CDCl₃, 200 MHz) δ=1.0 (t, 3H), 1.2 (t, 3H), 1.4 (t, 3H), 1.4 (t, 3H), 1.5 (m, 2H), 1.8 (m, 6H), 2.0 (m, 6H), 2.3 (m, 2H), 2.6 (m, 1H), 2.9 (d, 1H), 3.4 (d/d, 1H), 3.4 (m, 1H), 5.1 (m, 1H), 7.7 (m, 2H), 8.0 (br.s, 2H) ppm.
50. [(5S)-2-Cyclopentyl-4-(1-propyl)-5-hydroxy-7-spirocyclobutyl-5,6,7,8-tetrahydroquinolin-3-yl]-(4-trifluoromethylphenyl)-methanone

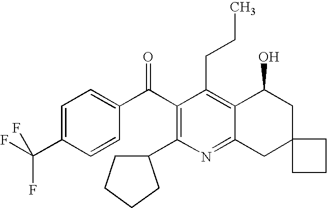

116 mg (0.25 mmol) of Example 35 are reacted analogously to the procedure of the compound from Example 36.

Yield: quantitative

¹H-NMR (CDCl₃, 200 MHz) δ=0.9 (t, 3H), 1.4 (m, 7H), 1.9 (m, 11H), 2.3 (m, 1H), 2.6 (m, 11H), 2.9 (d, 1H), 3.4 (d/d, 11H), 5.0 (m, 1H), 7.7 (m, 2H), 7.9 (m, 2H) ppm.

51. (5S)-2-Isopropyl-4-(4-fluorophenyl)-3-[(S)-hydroxy-(4-trifluoromethylphenyl)-methyl]-7-spirocyclobutyl-5,6,7,8-tetrahydroquinolin-5-ol (Anti Isomer)

52. (5S)-2-Isopropyl-4-(4-fluorophenyl)-3-[(R)-hydroxy-(4-trifluoromethylphenyl)-methyl]-7-spirocyclobutyl-5,6,7,8-tetrahydroquinolin-5-ol (Syn Isomer)

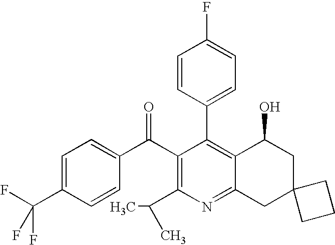

571 mg (1.15 mmol, 1 eq.) of Example 36 are introduced into 50 ml THF at 0° C., then 1.26ml (1.26 mmol, 1.1 eq.) of a one molar solution of lithium aluminium hydride in THF are added and the solution is stirred at 0° C. for one hour and overnight for 18 hours. It is then treated with 1 ml of methanol, and the solution is concentrated and chromatographed (silica gel, eluent cyclohexane/ethyl acetate mixtures).

Yield: 225 mg (39%) of anti isomer
294 mg (51%) of syn isomer
   Anti Isomer:

¹H-NMR (CDCl₃, 300 MHz) δ=0.8 (d, 3H), 1.2 (d, 3H), 1.4 (d, 1H), 2.0 (m, 6H), 2.1 (m, 1H), 2.2 (d, 1H), 2.3 (m, 1H), 2.9 (d, 1H), 3.0 (sept., 1H), 3.4 (d, 1H), 4.6 (t/d, 1H), 5.7 (d, 1H), 7.1 (m, 3H), 7.3 (m, 3H), 7.5 (m, 2H) ppm.

Syn Isomer:

¹H-NMR (CDCl₃, 300 MHz) δ=0.7 (d, 3H), 1.2 (d, 3H), 1.3 (d, 1H), 1.9 (m, 6H), 2.1 (m, 1H), 2.2 (d, 1H), 2.3 (m, 1H), 2.9 (d, 1H), 3.0 (sept., 1H), 3.4 (d, 1H), 4.6 (t/d, 1H), 5.7 (d, 1H), 7.1 (m, 3H), 7.3 (m, 3H), 7.5 (m, 2H) ppm.

53. (5S)-2-Isopropyl-4-phenyl-3-[(S)-hydroxy-(4-trifluoromethylphenyl)-methyl]-7-spirocyclobutyl-5,6,7,8-tetrahydroquinolin-5-ol (Anti Isomer)

54. (5S)-2-Isopropyl-4-phenyl-3-[(R)-hydroxy-(4-trifluoromethylphenyl)-methyl]-7-spirocyclobutyl-5,6,7,8-tetrahydroquinolin-5-ol (Syn Isomer)

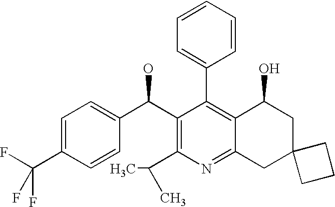

233 mg (0.49 mmol) of Example 38 are reacted analogously to the procedure of the compound from Example 51/52.

Yield: 61 mg (26%) of anti isomer
127 mg (54%) of syn isomer
   Anti Isomer:

¹H-NMR (CDCl₃, 300 MHz) δ=0.8 (d, 3H), 1.2 (d, 3H), 1.5 (d, 1H), 2.0 (m, 6H), 2.1 (m, 1H), 2.2 (d, 1H), 2.2 (m, 1H), 2.9 (d, 1H), 3.0 (sept., 1H), 3.4 (d, 1H), 4.7 (t/d, 1H), 5.7 (d, 1H), 7.1 (m, 1H), 7.3 (m, 6H), 7.5 (m, 2H) ppm.

Syn Isomer:

¹H-NMR (CDCl₃, 300 MHz) δ=0.7 (d, 3H), 1.2 (d, 3H), 1.4 (d, 1H), 2.0 (m, 6H), 2.1 (m, 1H), 2.2 (d, 1H), 2.2 (m, 1H), 2.9 (d, 1H), 3.0 (sept., 1H), 3.4 (d/d, 1H), 4.7 (t/d, 1H), 5.7 (d, 1H), 7.2 (m, 1H), 7.3 (m, 3H), 7.4 (m, 3H), 7.5 (m, 2H) ppm.

55. (5S)-2-Cyclopentyl-4-phenyl-3-[(S)-hydroxy-(4-trifluoromethylphenyl)-methyl]-7-spirocyclobutyl-5,6,7,8-tetrahydroquinolin-5-ol (Anti Isomer)

56. (5S)-2-Cyclopentyl-4-phenyl-3-[(R)-hydroxy-(4-trifluoromethylphenyl)-methyl]-7-spirocyclobutyl-5,6,7,8-tetrahydroquinolin-5-ol (Syn Isomer)

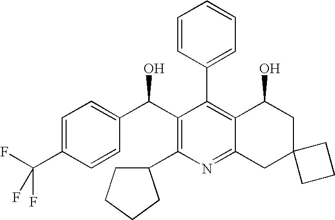

58 mg (0.11 mmol) of Example 39 are reacted analogously to the procedure of the compound from Example 51/52.

Yield: 20 mg (33.5%) of anti isomer
33 mg (56.7%) of syn isomer
   Anti Isomer:

¹H-NMR (CDCl₃, 300 MHz) δ=1.0 (m, 1H), 1.3 (m, 2H), 1.5 (d, 1H), 1.7 (m, 2H), 1.8 (m, 1H), 1.9 (m, 7H), 2.0 (m, 1H), 2.1 (d, 1H), 2.2 (m, 1H), 2.9 (d, 1H), 3.1 (m, 1H), 3.3 (d, 1H), 4.7 (t/d, 1H), 5.7 (d, 1H), 7.1 (m, 1H), 7.3 (m, 6H), 7.5 (m, 2H) ppm.

Syn Isomer:

¹H-NMR (CDCl₃, 300 MHz) δ=0.9 (m, 1H), 1.3 (m, 2H), 1.4 (d, 1H), 1.6 (m, 2H), 1.7 (m, 1H), 1.9 (m, 7H), 2.0 (m, 1H), 2.2 (d, 1H), 2.2 (m, 1H), 2.9 (d, 1H), 3.2 (m, 1H), 3.3 (d, 1H), 4.7 (t/d, 1H), 5.7 (d, 1H), 7.2 (m, 1H), 7.3 (m, 3H), 7.4 (m, 3H), 7.5 (m, 2H) ppm.

57. (5S)-2-Isopropyl-4-(3-thienyl)-3-[(S)-hydroxy-(4-trifluoromethylphenyl)-methyl]-7-spirocyclobutyl-5,6,7,8-tetrahydroquinolin-5-ol (Anti Isomer)

58. (5S)-2-Isopropyl-4-(3-thienyl)-3-[(R)-hydroxy-(4-trifluoromethylphenyl)-methyl]-7-spirocyclobutyl-5,6,7,8-tetrahydroquinolin-5-ol (Syn Isomer)

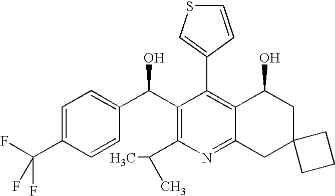

546 mg (1.12 mmol) of Example 40 are reacted analogously to the procedure of the compound from Example 51/52.

Yield: 186 mg (33.9%) of anti isomer (2 rotamers)
309 mg (56.3%) of syn isomer (2 rotamers)
   Anti Isomer

¹H-NMR (CDCl₃, 200 MHz) δ=0.7 (d, 3H), 0.8 (d, 3H), 1.2 (d, 3H), 1.2 (d, 3H), 1.7 (d, 1H), 2.0 (m, 6H), 2.1 (m, 1H), 2.2 (m, 1H), 2.9 (d, 1H rotamer 1), 2.9 (d, 1H rotamer 2), 3.0 (sept, 1H rotamer 1), 3.1 (d, 1H rotamer 2), 3.3 (d, 1H rotamer 1), 3.4 (d, 1H rotamer 2), 4.7 (t/d, 1H rotamer 1), 4.8 (t/d, 1H rotamer 2), 5.7 (d, 1H rotamer 1), 5.8 (d, 1H rotamer 2), 6.8 (m, 1H, rotamer 1), 7.1 (m, 1H), 7.3 (m, 3H, m, 1H rotamer 2), 7.5 (m, 2H) ppm.

Syn Isomer:

¹H-NMR (CDCl₃, 200 MHz) δ=0.7 (d, 3H), 0.7 (d, 3H), 1.2 (d, 3H), 1.2 (d, 3H), 1.4 (d, 1H), 1.6 (d, 1H), 2.0 (m, 6H), 2.1 (m, 1H), 2.2 (m, 1H), 2.9 (d, 1H rotamer 1), 2.9 (d, 1H rotamer 2), 3.1 (sept, 1H rotamer 1), 3.1 (d, 1H rotamer 2), 3.3 (d, 1H rotamer 1), 3.4 (d, 1H rotamer 2), 4.6 (t/d, 1H rotamer 1), 4.7 (t/d, 1H rotamer 2), 5.8 (d, 1H rotamer 1), 5.8 (d, 1H rotamer 2), 6.8 (m, 1H, rotamer 1), 7.0 (m, 1H rotamer 1), 7.0 (m, 1H rotamer 2), 7.1 (m, 1H rotamer 1), 7.2 (m, 2H, m, 1H rotamer 2), 7.4 (m, 1H), 7.5 (m, 2H) ppm.

59. (5S)-2-Cyclopentyl-4-(3-thienyl)-3-[(S)-hydroxy-(4-trifluoromethylphenyl)-methyl]-7-spirocyclobutyl-5,6,7,8-tetrahydroquinolin-5-ol (Anti Isomer)

60. (5S)-2-Cyclopentyl-4-(3-thienyl)-3-[(R)-hydroxy-(4-trifluoromethylphenyl)-methyl]-7-spirocyclobutyl-5,6,7,8-tetrahydroquinolin-5-ol (Syn Isomer)

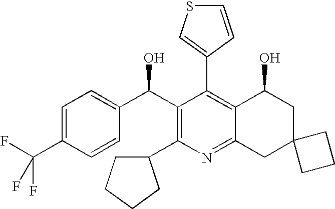

180 mg (0.35 mmol) of Example 41 are reacted analogously to the procedure of the compound from Example 51/52.

Yield: 47 mg (26.0%) of anti isomer (2 rotamers)
120 mg (66.4%) of syn isomer (2 rotamers)
   Anti Isomer

¹H-NMR (CDCl₃, 300 MHz) δ=0.9 (m, 1H), 1.3 (m, 2H), 1.5 (d, 1H), 1.7 (d, 1H), 1.7 (m, 3H), 1.8 (m, 2H), 1.9 (m, 6H), 2.1 (m, 1H), 2.2 (m, 1H), 3.3 (d, 1H rotamer 1), 2.9 (d, 1H, rotamer 2), 3.1 (m, 1H), 3.3 (d, 1H rotamer 1), 3.3 (d, 1H rotamer 2), 4.6 (t/d, 1H rotamer 1), 4.8 (t/d, 1H rotamer 2), 5.8 (d, 1H rotamer 1), 5.9 (d, 1H rotamer 2), 6.8 (m, 1H rotamer 1), 7.0 (m, 1H rotamer 2), 7, 1 (m, 1H rotamer 1), 7.3 (m, 3H), 7.4 (m, 1H rotamer 2), 7.5 (m, 2H) ppm.

Syn Isomer:

¹H-NMR (CDCl₃, 300 MHz) δ=1.0 (m, 1H), 1.3 (m, 2H), 1.4 (d, 1H), 1.6 (d, 1H), 1.6 (m, 3H), 1.9 (m, 8H), 2.1 (m, 1H), 2.2 (m, 1H), 2.9 (d, 1H rotamer 1), 2.9 (d, 1H, rotamer 2), 3.2 (m, 1H), 3.3 (d, 1H rotamer 1), 3.3 (d, 1H rotamer 2), 4.6 (t/d, 1H rotamer 1), 4.7 (t/d, 1H rotamer 2), 5.8 (d, 1H rotamer 1), 5.8 (d, 1H rotamer 2), 6.9 (m, 1H rotamer 1), 7.0 (m, 1H rotamer 2), 7.1 (m, 1H rotamer 1), 7.2 (m, 1H rotamer 2), 7.3 (m, 2H), 7.4 (m, 1H), 7.5 (m, 2H) ppm.

61. (5S)-2-Isopropyl-4-(2-thienyl)-3-[(S)-hydroxy-(4-trifluoromethylphenyl)-methyl]-7-spirocyclobutyl-5,6,7,8-tetrahydroquinolin-5-ol (Anti Isomer)

62. (5S)-2-Isopropyl-4-(2-thienyl)-3-[(R)-hydroxy-(4-trifluoromethylphenyl)-methyl]-7-spirocyclobutyl-5,6,7,8-tetrahydroquinolin-5-ol (Syn Isomer)

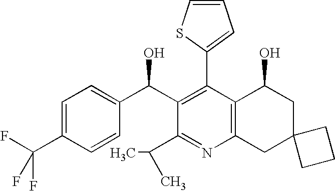

380 mg (0.78 mmol) of Example 42 are reacted analogously to the procedure of the compound from Example 51/52.

Yield: 80 mg (21.0%) of anti isomer
250 mg (65.5%) of syn isomer
   Anti Isomer:

¹H-NMR (CDCl₃, 300 MHz) δ=0.7 (d, 3H), 1.2 (d, 3H), 2.0 (m, 6H), 2.2 (m, 2H), 2.9 (d, 1H), 3.1 (m, 1H), 3.4 (d, 1H), 4.9 (br.s, 1H), 5.8 (br. s, 1H), 7.1 (m, 2H), 7.4 (m, 3H), 7.6 (m, 2H) ppm.

Syn Isomer:

¹H-NMR (CDCl₃, 300 MHz) δ=0.7 (d, 3H), 1.2 (d, 3H), 2.0 (m, 6H), 2.3 (m, 2H), 2.9 (d, 1H), 3.1 (sept, 1H), 3.4 (d, 1H), 4.8 (br.s, 1H), 5.8 (d, 1H), 7.1 (m, 2H), 7.3 (m, 2H), 7.4 (m, 1H), 7.6 (m, 2H) ppm.

63. (5S)-2-Cyclopentyl-4-(2-thienyl)-3-[(S)-hydroxy-(4-trifluoromethylphenyl)-methyl]-7-spirocyclobutyl-5,6,7,8-tetrahydroquinolin-5-ol (Anti Isomer)

64. (5S)-2-Cyclopentyl-4-(2-thienyl)-3-[(R)-hydroxy-(4-trifluoromethylphenyl-methyl]-7-spirocyclobutyl-5,6,7,8-tetrahydroquinolin-5-ol (Syn Isomer)

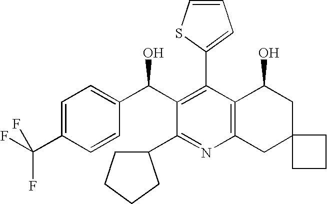

80 mg (0.16 mmol) of Example 43 are reacted analogously to the procedure of the compound from Example 51/52.

Yield: 21 mg (26%) of anti isomer
48 mg (59%) of syn isomer
   Anti Isomer:

LC/MS (A) rt 2.72 min, MS (ESI): 514 [M+H]

Syn Isomer:

LC/MS (A) rt 2.82 min, MS (ESI): 514 [M+H]

65. (5S)-2-Isopropyl-4-cyclohexyl-3-[(S)-hydroxy-(4-trifluoromethylphenyl)-methyl]-7-spirocyclobutyl-5,6,7,8-tetrahydroquinolin-5-ol (Anti Isomer)

66. (5S)-2-Isopropyl-4-cyclohexyl-3-[(R)-hydroxy-(4-trifluoromethylphenyl)-methyl]-7-spirocyclobutyl-5,6,7,8-tetrahydroquinolin-5-ol (Syn Isomer)

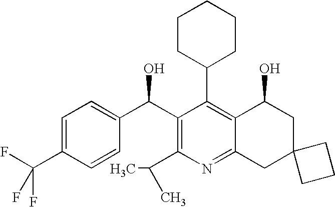

345 mg (0.71 mmol) of Example 44 are reacted analogously to the procedure of the compound from Example 51/52.

Yield: 94 mg (27%) of anti isomer
204 mg (59%) of syn isomer
   Anti Isomer:

¹H-NMR (CDCl₃, 300 MHz) δ=0.6 (d, 3H), 1.1 (d, 3H), 1.4 (m, 3H); 1.5 (d, 1H), 1.9 (m, 13H), 2.2 (m, 3H), 2.8 (d, 1H), 2.9 (sept, 1H), 3.3 (d, 1H), 3.5 (br.m, 1H), 5.1 (t/d, 1H), 6.6 (br.s, 1H), 7.4 (m, 2H), 7.6 (m, 2H) ppm.

Syn Isomer:

¹H-NMR (CDCl₃, 300 MHz) δ=0.6 (d, 3H), 1.1 (d, 3H), 1.2 (m, 2H); 1.5 (m, 2H), 1.9 (m, 13H), 2.2 (m, 3H), 2.8 (d, 1H), 2.9 (sept, 1H), 3.3 (d, 1H), 3.5 (br.m, 1H), 5.1 (t/d, 1H), 6.7 (br.s, 1H), 7.4 (m, 2H), 7.6 (m, 2H) ppm.

67. (5S)-2-Cyclopentyl-4-cyclohexyl-3-[(S)-hydroxy-(4-trifluoromethylphenyl)-methyl]-7-spirocyclobutyl-5,6,7,8-tetrahydroquinolin-5-ol (Anti Isomer)

68. (5S)-2-Cyclopentyl-4-cyclohexyl-3-[(R)-hydroxy-(4-trifluoromethylphenyl)-methyl]-7-spirocyclobutyl-5,6,7,8-tetrahydroquinolin-5-ol (Syn Isomer)

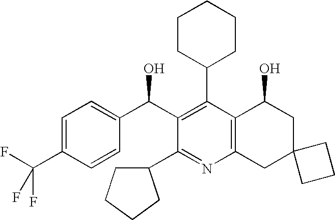

774 mg (0.51 mmol) of Example 45 are reacted analogously to the procedure of the compound from Example 51/52.

Yield: 72 mg (27.6%) of anti isomer
180 mg (69.0%) of syn isomer
   Anti Isomer:

¹H-NMR (CDCl₃, 200 MHz) δ=0.7 (m, 1H), 1.2 (m, 5H), 1.5 (d, 1H), 1.9 (m, 18H), 2.2 (m, 3H), 2.8 (d, 1H), 3.0 (m, 1H), 3.3 (d, 1H), 3.5 (m, 1H), 5.1 (m, 1H), 6.7 (br.d, 1H), 7.4 (m, 2H), 7.6 (m, 2H) ppm.

Syn Isomer:

¹H-NMR (CDCl₃, 300 MHz) δ=0.6 (m, 1H), 1.2 (m, 5H), 1.4 (d, 1H), 1.7-2.1 (compl. region, 18H), 2.2 (m, 3H), 2.8 (d, 1H), 3.0 (m, 1H), 3.3 (d/d, 1H), 3.5 (m, 1H), 5.1 (m, 1H), 6.7 (br.d, 1H), 7.4 (m, 2H), 7.6 (m, 2H) ppm.

69. (5S)-2-Isopropyl-4-cyclopentyl-3-[(S)-hydroxy-(4-trifluoromethylphenyl)-methyl]-7-spirocyclobutyl-5,6,7,8-tetrahydroquinolin-5-ol (Anti Isomer)

70. (5S)-2-Isopropyl-4-cyclopentyl-3-[(R)-hydroxy-(4-trifluoromethylphenyl)-methyl]-7-spirocyclobutyl-5,6,7,8-tetrahydroquinolin-5-ol (Syn Isomer)

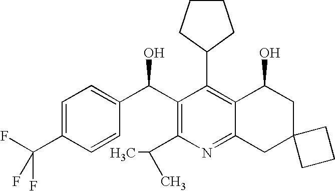

237 mg (0.50 mmol) of Example 46 are reacted analogously to the procedure of the compound from Example 51/52.

Yield: 116 mg (49.0%) of anti isomer
102 mg (42.7%) of syn isomer
   Anti Isomer:

¹H-NMR (CDCl₃, 200 MHz) δ=0.7 (d, 3H), 1.1 (d, 3H), 1.5 (d, 1H), 1.7 (m, 3H), 1.9 (m, 10H), 2.1 (m, 2H), 2.2 (d, 1H), 2.3 (m, 1H), 2.9 (d, 1H), 2.9 (sept, 1H), 3.3 (d/d, 1H), 3.8 (m, 1H), 5.1 (t/d, 1H), 6.2 (d, 1H), 7.4 (m, 2H), 7.6 (m, 2H) ppm.

Syn Isomer:

¹H-NMR (CDCl₃, 300 MHz) δ=0.7 (d, 3H), 1.1 (d, 3H), 1.5 (d, 1H), 1.7 (m, 3H), 1.9 (m, 10H), 2.1 (m, 1H), 2.2 (m, 3H), 2.8 (d, 1H), 2.9 (m, 1H), 3.3 (d/d, 1H), 3.8 (m, 1H), 5.1 (t/d, 1H), 6.2 (d, 1H), 7.4 (m, 2H), 7.6 (m, 2H) ppm.

71. (5S)-2,4-Dicyclopentyl-3-[(S)-hydroxy-(4-trifluoromethylphenyl)-methyl]-7-spirocyclobutyl-5,6,7,8-tetrahydroquinolin-5-ol (Anti Isomer)

72. (5S)-2,4-Dicyclopentyl-3-[(R)-hydroxy-(4-trifluoromethylphenyl)-methyl]-7-spirocyclobutyl-5,6,7,8-tetrahydroquinolin-5-ol (Syn Isomer)

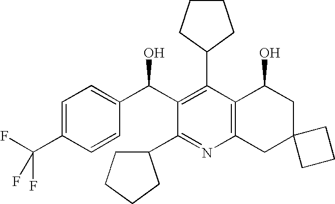

154 mg (0.31 mmol) of Example 47 are reacted analogously to the procedure of the compound from Example 51/52.

Yield: 65 mg (41.8%) of anti isomer
46 mg (29.6%) of syn isomer
   Anti Isomer:

¹H-NMR (CDCl₃, 300 MHz) δ=0.9 (m, 1H), 1.3 (m, 2H), 1.5 (d, 1H), 1.7 (m, 9H), 1.9 (m, 9H), 2.1 (m, 2H), 2.2 (d, 1H), 2.3 (m, 1H), 2.8 (d, 1H), 3.0 (m, 1H), 3.3 (d/d, 1H), 3.8 (m, 1H), 5.1 (t/d, 1H), 6.2 (d, 1H), 7.4 (m, 2H), 7.6 (m, 2H) ppm.

Syn Isomer:

¹H-NMR (CDCl₃, 300 MHz) δ=0.9 (m, 1H), 1.3 (m, 2H), 1.5 (d, 1H), 1.7-2.0 (compl. region, 18H) 2.1 (m, 2H), 2.3 (m, 4H), 2.8 (d, 1H), 3.0 (m, 1H), 3.3 (d/d, 1H), 3.8 (m, 1H), 5.1 (t/d, 1H), 6.2 (d, 1H), 7.4 (m, 2H), 7.6 (m, 2H) ppm.

73. (5S)-2-Cyclopentyl-4-cyclobutyl-3-[(S)-hydroxy-(4-trifluoromethylphenyl)-methyl]-7-spirocyclobutyl-5,6,7,8-tetrahydroquinolin-5-ol (Anti Isomer)

74. (5S)-2-Cyclopentyl-4-cyclobutyl-3-[(R)-hydroxy-(4-trifluoromethylphenyl)-methyl]-7-spirocyclobutyl-5,6,7,8-tetrahydroquinolin-5-ol (Syn Isomer)

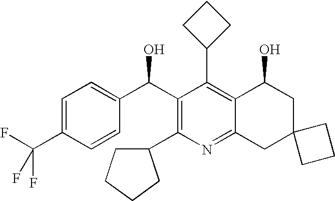

346 mg (0.72 mmol) of Example 48 are reacted analogously to the procedure of the compound from Example 51/52.

Yield: 166 mg (47.9%) of anti isomer
57 mg (16.5%) of syn isomer
   Anti Isomer:

¹H-NMR (CDCl₃, 200 MHz) δ=0.7 (m, 1H), 1.2 (m, 2H), 1.5 (d, 1H), 1.7 (m, 2H), 1.7-2.1 (compl. region., 14H), 2.3 (d, 1H), 2.5 (m, 3H), 2.9 (d, 1H), 3.1 (m, 1H), 3.1 (d, 1H), 4.3 (m, 1H), 5.2 (t/d, 1H), 6.6 (d, 1H), 7.3 (m, 2H), 7.5 (m, 2H) ppm.

Syn Isomer:

LC/MS (A) rt 2.32 min, MS (ESI): 486 [M+H].

75. (5S)-2-Cyclopentyl-4-isopropyl-3-[(S)-hydroxy-(4-trifluoromethylphenyl)-methyl]-7-spirocyclobutyl-5,6,7,8-tetrahydroquinolin-5-ol (Anti Isomer)

76. (5S)-2-Cyclopentyl-4-isopropyl-3-[(R)-hydroxy-(4-trifluoromethylphenyl)-methyl]-7-spirocyclobutyl-5,6,7,8-tetrahydroquinolin-5-ol Syn Isomer)

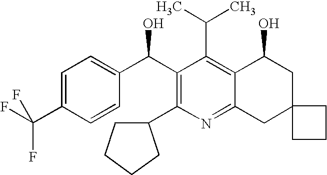

83 mg (0.18 mmol) of Example 49 are reacted analogously to the procedure of the compound from Example 51/52.

Yield: 26 mg (30.7%) of anti isomer
16 mg (18.8%) of syn isomer
   Anti Isomer:

LC/MS (A) rt 2.17 min, MS (ESI): 474 [M+H]

Syn Isomer:

LC/MS (A) rt 2.24 min, MS (ESI): 474 [M+H]
77. (5S)-2-Cyclopentyl-4-(1-propyl)-3-[(S)-hydroxy-(4-trifluoromethylphenyl)-methyl]-7-spirocyclobutyl-5,6,7,8-tetrahydroquinolin-5-ol (Anti Isomer)

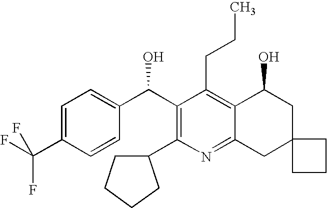

109 mg (0.23 mmol) of Example 50 are reacted analogously to the procedure of the compound from Example 51/52.

Yield: 56 mg (51.4%) of anti isomer

¹H-NMR (CDCl₃, 200 MHz) δ=1.0 (m, 4H), 1.2 (m, 4H), 1.5 (m, 4H), 1.9 (m, 10H), 2.2 (m, 3H), 2.8 (d, 1H), 3.1 (m, 1H), 3.4 (m, 1H), 5.1 (t/d, 1H), 6.3 (d, 1H), 7.4 (m, 2H), 7.6 (m, 2H) ppm.
78. [(5S)-5-tert-Butyldimethylsilanyloxy-2-isopropyl-4-(4-fluorophenyl)-7-spirocyclobutyl-5,6,7,8-tetrahydroquinolin-3-yl]-(4-trifluoromethylphenyl)-methanone

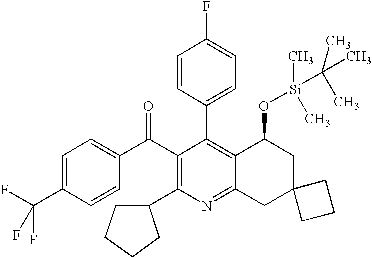

735 mg (1.40 mmol) of ketoalcohol from Example 37 are introduced into toluene (5 ml, p.a., dried over molecular sieve) under argon, 600 mg (5.60 mmol) of 2,6-lutidine are added at RT and the mixture is cooled to −16° C. 740 mg (2.81 mmol) of tert-butyldimethylsilyl trichloromethanesulphonate in toluene (1.5 ml) are added dropwise to this solution and it is washed twice with 0.25 ml of toluene each time. After 15 min, it is warmed to 0° C. and the reaction mixture is stirred at this temperature for 80 min. For work-up, 0.1N hydrochloric acid (20 ml) is added and, after warming to RT, the mixture is extracted by shaking with ethyl acetate. The aqueous phase is extracted with ethyl acetate a further three times, the combined organic phases are washed with a 1:1 mixture of sodium hydrogencarbonate solution and saturated sodium chloride solution and this aq. phase is in turn extracted with ethyl acetate. The combined organic phases are dried over sodium sulphate, filtered and concentrated in vacuo. The residue is dissolved in ethyl acetate/petroleum ether and a little dichloromethane and purified by chromatography on silica gel using ethyl acetate/petroleum ether 1:20. 889 mg (99% of theory) of a colourless hard foam are obtained.

Rf (EA/PE 1:9)=0.56

MS (FAB): 638 (M+H)

¹H-NMR (300 MHz, CDCl₃) δ[ppm]: −0.65 (br. s, 3H), −0.07 (s, 3H), 0.71 (s, 9H), 1.41-2.11 (m, 14H), 2.17 (dd, 1H, J1=14.1 Hz, J2=3.2 Hz), 2.20-2.31 (m, 1H), 2.82 (br. m, 1H), 3.04 (d, 1H, J=16.4 Hz), 3.45 (d, 1H, J=16.4 Hz), 4.96 (br. s, 1H), 6.60-7.20 (br. m, 4H), 7.55 (br. m, 4H).
79. [(5 S)-5-tert-Butyldimethylsilanyloxy-2-isopropyl-3 [(S)-hydroxy-(4-trifluoromethylphenyl)-methyl]-4-(4-fluorophenyl)-7-spirocyclobutyl-5,6,7,8-tetrahydroquinoline

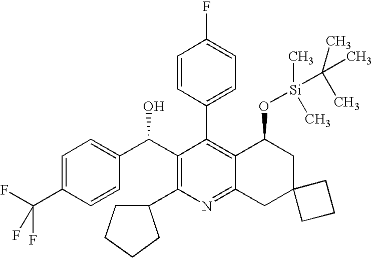

828 mg (1.30 mmol) of silyloxy ketone from Example 78 are introduced into toluene under argon (5 ml, p.a., dried over molecular sieve) with cooling in an ice bath and 1.50 g (5.19 mmol) of RedA1* solution 70% in toluene are added dropwise. The reaction mixture is stirred for 1.5 h with ice cooling, and for 45 min with slow warming to 13° C. and 50 min without cooling. To terminate the reaction, it is again cooled to 0° C. and methanol (1 ml) is added. After evolution of gas is complete, it is extracted by shaking with ethyl acetate and a mixture of aq. sodium hydrogencarbonate solution and sat. sodium chloride solution. The aq. phase is extracted a further three times with ethyl acetate, and the combined org. phases are dried over sodium sulphate, filtered and concentrated in vacuo. The residue (878 mg) is purified by chromatography on silica gel using ethyl acetate/petroleum ether 1:20. 173 mg (21% of theory) of the epimeric alcohol (syn configuration) are obtained as a hard foam and after fresh chromatography 607 mg (73% of theory) of the desired alcohol are obtained as a crystalline solid.

sodium bis-(2-methoxyethoxy)aluminium dihydride

Anti Isomer:

Rf (EA/PE 1:9)=0.22

MS (ESI pos): 640 (M+H)

¹H-NMR (300 MHz, CDCl₃) δ[ppm]: −0.53 (s, 3H), −0.05 (s, 3H), 0.77 (s, 9H), 1.09-2.28 (m, 17H), 2.97 (d, 1H, J=16.2 Hz), 3.09 (quint., 1H), 3.39 (d, 1H, J=16.2 Hz), 4.77 (t, 1H), 5.67 (br. d, 1H), 6.88-7.08 (m, 3H), 7.09-7.19 (m, 1H), 7.29 (d, 2H), 7.53 (d, 2H).

Syn Isomer:

Rf (EA/PE 1:9)=0.31

MS (ESI pos): 640 (M+H).
80. (5S)-2-Cyclopentyl-4-(4-fluorophenyl)-3-[(S)-hydroxy-(4-trifluoromethylphenyl)-methyl]-7-spirocyclobutyl-5,6,7,8-tetrahydroquinolin-5-ol

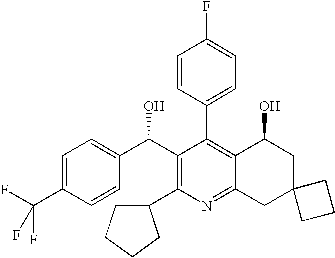

30 mg (0.05 mmol) of Example 79 are introduced under argon and treated with 1M TBAF solution in THF (0.5 ml). The reaction mixture is stirred overnight at RT. After addition of sat. sodium hydrogencarbonate soln, it is extracted three times with EA, the combined org. phases are dried over sodium sulphate and the solvent is removed in vacuo. The residue (51 mg) is purified by flash chromatography on silica gel using EA/CH 1:4. A colourless hard foam is isolated (23 mg; 94% of theory).

Rf (EA/CH 1:4)=0.26

MS (ESI): 526 (M+H)

¹H-NMR (300 MHz, CDCl₃) δ=1.0-2.3 (m, 17 H); 2.14 (m, 1H); 2.88 (d, 1H); 3.13 (m, 1H); 3.35 (d, 1H); 4.60 (m, 1H); 5.74 (d, 1H); 6.97-7.11 (m, 3H); 7.20-7.35 (m, 3H); 7.48-7.56 (m, 2H) ppm.

A. CETP Inhibition Testing

A1. Obtainment of CETP

CETP is obtained in partially purified form from human plasma by differential centrifugation and column chromatography and used for the test. To this end, human plasma is adjusted to a density of 1.21 g per ml using NaBr and centrifuged at 4° C. at 50000 rpm for 18 h. The bottom fraction (d>1.21 g/ml) is applied to a Sephadex® Phenyl-Sepharose 4B (Pharmacia) column, washed with 0.15 M NaCl/0.001 M tris HCl pH 7.4 and then eluted with dist. water. Die CETP-active fractions are pooled, dialysed against 50 mM Na acetate pH 4.5 and applied to a CM-Sepharose® (Pharmacia) column. The mixture is then eluted using a linear gradient (0-1 M NaCl). The pooled CETP fractions are dialysed against 10 mM TrisHCl pH 7.4 and then further purified by chromatography on a Mono Q® column (Pharmacia).

A2. CETP Fluorescence Test

Measurement of the CETP-catalysed transfer of a fluorescent cholesterol ester between liposomes—modified according to the procedure of Bisgaier et al., J. Lipid Res. 34, 1625 (1993).

For the production of the donor liposomes, 1 mg of cholesteryl 4,4-difluoro-5,7-dimethyl-4-bora-3a,4a-diaza-s-indacene-3-dodecanoate (cholesteryl BODIPY® FL C₁₂, Molecular Probes) is dissolved in 600 μl of dioxane with 5.35 mg of triolein and 6.67 mg of phosphatidylcholine with gentle warming in an ultrasonic bath and this solution is added very slowly with ultrasonication to 63 ml of 50 mM tris/HCl, 150 mM NaCl, 2 mM EDTA buffer pH 7.3 at RT.

The suspension is then ultrasonicated under an N₂ atmosphere for 30 minutes in the Braukson ultrasonic bath at about 50 watts, the temperature being kept at about 20° C.

The acceptor liposomes are obtained analogously from 86 mg of cholesteryl oleate, 20 mg of triolein and 100 mg of phosphatidylcholine dissolved in 1.2 ml of dioxane and 114 ml of above buffer by ultrasonication at 50 watts (20° C.) for 30 minutes.

For testing, a test mix consisting of 1 part of above buffer, 1 part of donor liposomes and 2 parts of acceptor liposomes is used.

80 μl of test mix are treated with 1-3 μg of enriched CETP fraction, obtained from human plasma by means of hydrophobic chromatography, and 2 μl of the substance to be investigated in DMSO and incubated at 37° C. for 4 hours.

The change in the fluorescence at 485/535 nm is a measure of the CE transfer; the inhibition of the transfer in comparison to the control batch without substance is determined.

The following table gives the results for the examples:

| Ex- | | IC₅₀ |
|---|---|---|
| am- | | (nM) |
| ple | | Fluor. |
| No. | Structure | test |
| 80 | 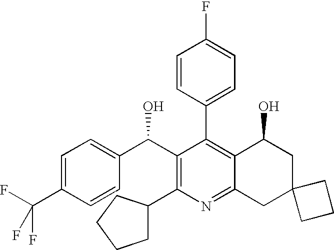 | 9 |
| 59 | 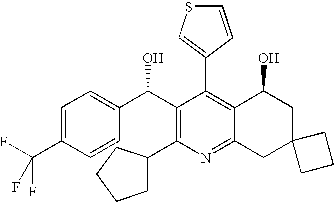 | 60 |
| 67 | 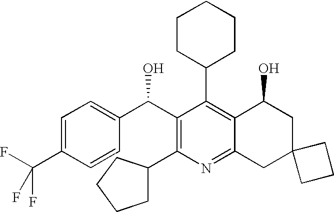 | 60 |
| 71 | 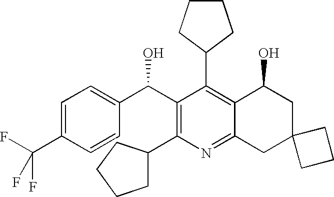 | 40 |
| 55 | 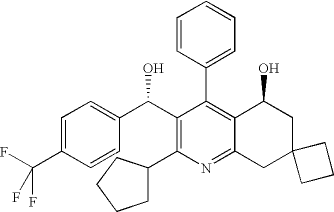 | 65 |
| 77 | 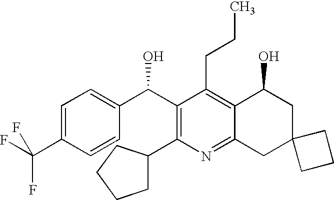 | 2000 |
| 63 | 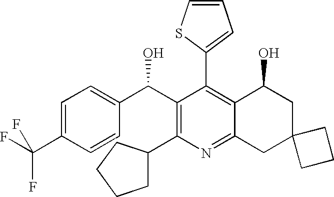 | 70 |
| 75 | 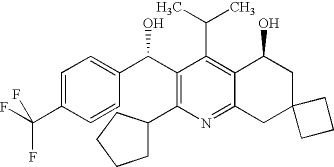 | 70 |
| 73 | 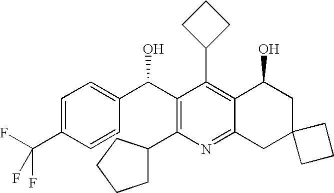 | 800 |
| 61 | 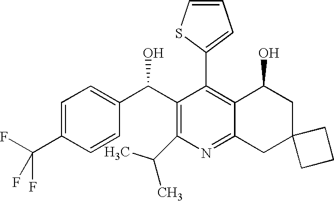 | 30 |
| 57 | 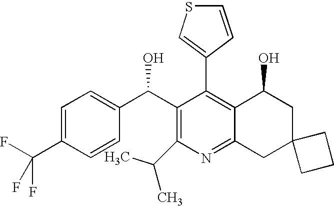 | 45 |
| 53 | 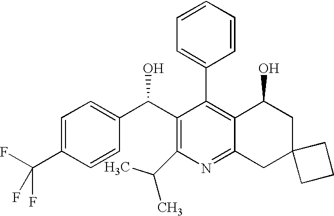 | 30 |
| 51 | 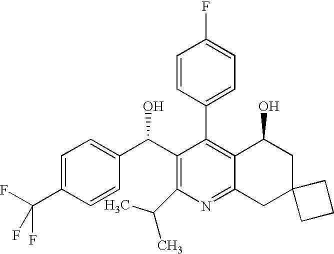 | 12 |
| 65 | 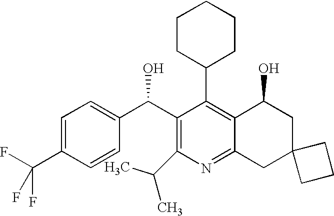 | 80 |
| 69 | 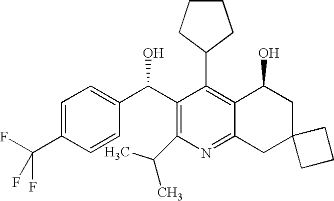 | 70 |

A3. Obtainment of Radiolabelled HDL

50 ml of fresh human EDTA plasma are adjusted to a density of 1.12 using NaBr and centrifuged at 4° C. in a Ty 65 rotor at 50000 rpm for 18 h. The upper phase is used for the obtainment of cold LDL. The lower phase is dialysed against 3×41 of PDB buffer (10 mM tris/HCl pH 7.4, 0.15 mM NaCl, 1 mM EDTA, 0.02% NaN₃). Per 10 ml of retentate volume, 20 μl of 3H-cholesterol (Dupont NET-725; 1 μC/μl dissolved in ethanol!) is then added and the mixture is incubated at 37° C. under N₂ for 72 h.

The batch is then adjusted to the density 1.21 using NaBr and centrifuged at 20° C. in a Ty 65 rotor at 50000 rpm for 18 h. The upper phase is recovered and the lipoprotein fractions are purified by gradient centrifugation. To this end, the isolated, labelled lipoprotein fraction is adjusted to a density of 1.26 using NaBr. 4 ml each of this solution are covered in centrifuged tubes (SW 40 rotor) with a layer of 4 ml of a solution of density 1.21 and 4.5 ml of a solution of 1.063 (density solutions of PDB buffer and NaBr) and then centrifuged for 24 h at 38000 rpm and 20° C. in the SW 40 rotor. The intermediate layer lying between the density 1.063 and 1.21, containing the labelled HDL, is dialysed against 3×100 volumes of PDB buffer at 4° C.

The retentate contains radiolabelled ³H-CE-HDL, which, adjusted to about 5×10⁶ cmp per ml, is used for the test.

A4. CETP-SPA Test

For testing of the CETP activity, the transfer of ³H-cholesterol ester from human HD lipoproteins to biotinylated LD lipoproteins is measured.

The reaction is ended by addition of streptavidin-SPA®beads (Amersham) and the transferred radioactivity is determined directly in a liquid scintillation counter.

In the test batch, 10 μl of HDL-³H-cholesterol ester (˜50000 cpm) are incubated at 37° C. for 18 h with 10 μl of biotin-LDL (Amersham) in 50 mM Hepes/0.15 M NaCl/0.1% bovine serum albumin/0.05% NaN₃ pH 7.4 containing 10 μl of CETP (1 mg/ml) and 3 μl of solution of the substance to be tested (dissolved in 10% DMSO/1% RSA). 200 μl of the SPA-streptavidin bead solution (TRKQ 7005) are then added, incubated further with shaking for 1 h and then measured in a scintillation counter.

Corresponding incubations with 10 μl of buffer, 10 μl of CETP at 4° C. and 10 μl of CETP at 37° C. serve as controls.

The activity transferred in the control batches with CETP at 37° C. is rated as 100% transfer. The substance concentration at which this transfer is reduced to half is specified as the IC₅₀ value.

The following table gives the results for the examples:

| | IC₅₀ (nM) |
|---|---|
| Example No. | SPA test |
| 80 | 5 |
| 59 | 35 |
| 67 | 15 |
| 61 | 40 |
| 57 | 40 |
| 53 | 30 |
| 51 | 15 |

B1. Measurement of the Ex Vivo Activities on Transgenic hCETP Mice

To test for CETP-inhibitory activity, the substances are administered orally using a stomach tube to transgenic hCETP mice of our own breeding (Dinchuk et al. BBA (1995) 1295-301). To this end, male mice are randomly assigned to groups having an equal number of animals, as a rule n=3, one day before the start of the experiment. Before administration of the substance, blood is taken from each mouse by puncture of the retroorbital venous plexus for the determination of its basal CETP activity in the serum (T1). The test substance is then administered to the animals using the stomach tube. At specific times after administration of the test substance, blood is taken from the animals by puncture a second time (T2), as a rule 1, or 3 and 6 h after substance administration, but if appropriate this can also be carried out at another time.

In order to be able to assess the inhibitory activity of a substance, for each time, i.e. 1 or 3 or 6 h, a corresponding control group is employed whose animals only receive the formulating agent without substance. In the control animals, the second blood sampling per animal is carried out as in the substance-treated animals in order to be able to determine the change in the CETP activity without inhibitor over the corresponding experimental time interval (1, 3 or 6 h).

After termination of the clotting, the blood samples are centrifuged and the serum is removed by pipette.

For the determination of the CETP activity, the cholesteryl ester transport over 4 h is determined. To this end, as a rule 2 μl of serum are employed in the test batch and the test is carried out as described under “CETP fluorescence test”.

The differences in the cholesteryl ester transport (pM CE*/h (T2)−pM CE*/h (T1)) are calculated for each animal and averaged in the groups. A substance which at one of the times reduces the cholesteryl ester transport by >30% is regarded as active.

| | % inhibition at | | |
|---|---|---|---|
| | 30 mg/kg | | |
| Example No. | 1 h | 3 h | 6 h |
| 63 | 74 | 55 | 40 |
| 61 | 71 | 49 | 35 |
| 53 | 69 | 51 | 44 |
| 51 | 72 | 64 | 60 |
| 65 | 69 | 46 | 28 |

B2. Measurement of the In Vivo Activity in Syrian Golden Hamsters

In experiments for the determination of the oral action on lipoproteins and triglycerides, test substance dissolved in DMSO and 0.5% suspended in Tylose are administered perorally by means of a stomach tube to Syrian golden hamsters bred in-house. For the determination of the CETP activity, before the start of the experiment blood is taken by retro-orbital puncture (about 250 μl). The test substances are then administered perorally by means of a stomach tube. The control animals receive identical volumes of solvent without test substance. The feed is then withdrawn from the animals and blood is taken at various times—up to 24 hours after substance administration—by puncture of the retroorbital venous plexus.

Clotting is terminated by incubation at 4° C. overnight, then centrifugation is carried out for 10 minutes at 6000×g. The content of cholesterol and triglycerides in the serum thus obtained is determined with the aid of modified commercially obtainable enzymatic tests (Ecoline 25 Cholesterol 1.14830.0001 Merck Diagnostica, Ecoline 25 Triglycerides 1.14856.0001 Merck Diagnostica). Serum is suitably diluted using physiological saline solution.

10 μl of serum dilution are treated with 200 μl of Ecoline 25 reagent in 96-hole plates and incubated for 10 minutes at room temperature. The optical density is then determined at a wavelength of 490 nm using an automatic plate reader. The triglyceride or cholesterol concentration contained in the samples is determined with the aid of a standard curve measured in parallel.

The determination of the content of HDL cholesterol is carried out after precipitation of the ApoB-containing lipoproteins (Sigma 352-4 HDL cholesterol reagent) according to the manufacturer's instructions.

| Example | % HDL increase after 24 h |
|---|---|
| No. | (Dose: 2 × 10 mg/kg) |
| 80 | 17 |
| 71 | 14 |
| 53 | 19 |
| 51 | 17 |

B3. Measurement of the In Vivo Activity in Transgenic hCETP Mice

In experiments for the determination of the oral action on lipoproteins and triglycerides, test substance is administered to transgenic mice using a stomach tube (Dinchuck, Hart, Gonzalez, Karmann, Schmidt, Wirak; BBA (1995), 1295, 301). Before the start of the experiment, blood is taken from the mice retroorbitally in order to determine cholesterol and triglycerides in the serum. The serum is obtained as described above for hamsters by incubation at 4° C. overnight and subsequent centrifugation at 6000×g. After a week, blood is again taken from the mice in order to determine lipoproteins and triglycerides. The change in the parameters measured is expressed as the percentage change compared with the starting value.

| Example | % HDL increase after 4 d |
|---|---|
| No. | (Dose: 4 × 10 mg/kg) |
| 69 | 28 |
| 51 | 57 |

Abbreviations Used:
Cy=cyclohexane
EA=ethyl acetate
PE=petroleum ether
THF=tetrahydrofuran
DAST=dimethylaminosulphur trifluoride
PTS=para-toluenesulphonic acid
PDC=pyridinium dichromate
PE/EA=petroleum ether /ethyl acetate
Tol=toluene

The LC-MS values measured were determined according to the following methods:

LC-MS Method A

| | |
|---|---|
| LC parameter | Solution A acetonitrile |
| | Solution B 0.3 g of 30% HCl/l of water |
| | Column temperature 50° C.; |
| | Column Symmetry C18 2.1 × 150 mm |

Gradient:

| Time [min] | % A | % B | Flow [ml/min] |
|---|---|---|---|
| 0 | 10 | 90 | 0.9 |
| 3 | 90 | 10 | 1.2 |
| 6 | 90 | 10 | 1.2 |

LC-MS Method B

| | |
|---|---|
| LC parameter | Solution A acetonitrile/0.1% formic acid |
| | Solution B water/0.1% formic acid |
| | Column temperature 40° C.; |
| | Column Symmetry C18 2.1 × 50 mm |

Gradient:

| Time [min] | % A | % B | Flow [ml/min] |
|---|---|---|---|
| 0 | 10 | 90 | 0.5 |
| 4 | 90 | 10 | 0.5 |
| 6 | 90 | 10 | 0.5 |
| 6.1 | 10 | 90 | 1.0 |

1. A compound of the formula (I) 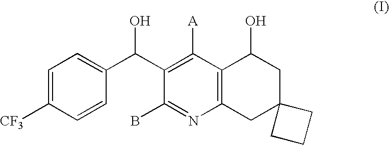
in which
A represents a radical 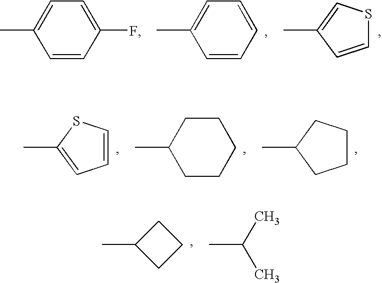
—(CH₂)₂CH₃ and
B represents a radical 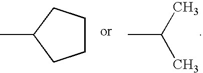
or a pharmaceutically acceptable salt thereof.

2. The compound according to claim 1, in which A represents para-fluorophenyl.

3. The compound according to claim 1, in which B represents isopropyl.

4. The compound according to claim 1 having the anti isomer form.

5. (Cancelled)

6. A pharmaceutical composition comprising a compound as defined in claim 1, 2, 3, or 4 and at least one inert, non-toxic, pharmaceutically suitable vehicle, solvent or excipient.

7. (Cancelled)

8. (Cancelled)

9. A method for inhibition of the cholesterol ester transfer protein (CETP) and for stimulation of reverse cholesterol transport, comprising administering an effective amount of a compound of claim 1, 2, 3, 4, 5, or 6.

10. A method for lowering the LDL cholesterol level in the blood and simultaneously increasing the HDL cholesterol level comprising administering an effective amount of a compound of claim 1, 2, 3, 4, 5, or 6.

11. A method for the treatment of hypolipoproteinaemia, dyslipidaemias, hypertriglyceridaemias, hyperlipidaemias, arteriosclerosis adiposity and obesity stroke or Alzheimer's disease, comprising administering an effective amount of a compound of claim 1.

12. (Cancelled)

13. A process for the preparation of compounds of the formula (I) as defined in claim 1, characterized in that a compound of the general formula (II) 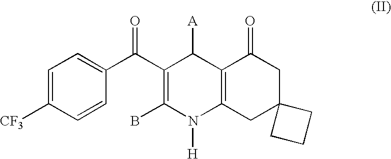
in which
A and B have the meanings indicated in claim 1,
is first oxidized to a compound of the general formula (III) 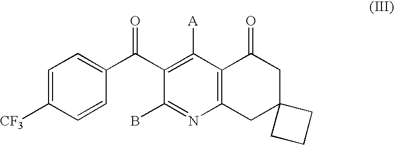
in which
A and B have the meanings indicated in claim 1,
these are reacted in a next step by means of an asymmetric reduction to give a compound of the general formula (IV) 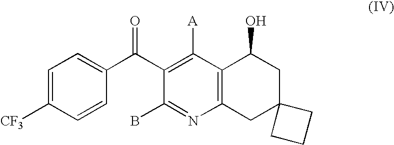
in which
A and B have the meanings indicated in claim 1,
this is then
(A) converted by the introduction of a hydroxy protective group into a compound of the general formula (V) 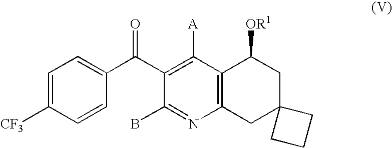
in which
R¹ represents a hydroxy protective group,
in which
R², R³ and R⁴ are identical or different and denote C₁-C₄-alkyl,
in a subsequent step the compound of the general formula (VI) 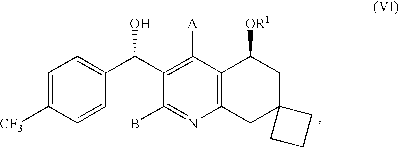
in which
R¹, A and B have the meanings indicated in claim 1,
is prepared from this by means of diastereoselective reduction
and finally the hydroxy protective group is cleaved according to customary methods,
or
(B) the compound of the formula (IV) is reduced directly.

14. The process of claim 13, in which R¹ of formula (V) is a radical of the formula —SiR²R³R⁴.
